(19) United States
(12) Patent Application Publication
(10) Pub. No.: US 2023/0357432 A1
(43) Pub. Date: Nov. 9, 2023

(54) PHARMACEUTICAL FORMULATION OF CONCIZUMAB AND METHOD OF PRODUCTION THEREOF

(71) Applicant: Novo Nordisk Health A/S, Bagsværd (DK)

(72) Inventors: Jonas Oldenburg Joergensen, Espergaerde (DK); Sarah Elizabeth Jones, Soeborg (DK)

(21) Appl. No.: 18/026,136

(22) PCT Filed: Sep. 23, 2021
(86) PCT No.: PCT/EP2021/076230

§ 371 Date: Mar. 14, 2023

(30) Foreign Application Priority Data
Sep. 24, 2020 (EP) 20197949.9

(51) Int. Cl. C07K 16/38; A61K 9/08; A61K 47/10; A61K 47/26; A61K 47/02

(52) U.S. Cl. CPC C07K 16/38; A61K 9/08; A61K 47/10; A61K 47/26; A61K 47/02

(57) ABSTRACT

Disclosed herein is a method of preparing a pharmaceutical formulation comprising a preservative, a surfactant and an anti-TFPI antibody (concizumab) as the active pharmaceutical ingredient. Application of the disclosed method results in the pharmaceutical formulation having improved chemical and physical purity. Also disclosed herein is the pharmaceutical formulation obtained using said method and its medical uses.

### TECHNICAL FIELD

Disclosed herein is a method of manufacturing a pharmaceutical formulation comprising an antibody, a preservative and a surfactant. Also disclosed herein is the pharmaceutical formulation obtained using said method and medical uses thereof.

### INCORPORATION-BY-REFERENCE OF THE SEQUENCE LISTING

The present application is filed with a Sequence Listing in electronic form. The contents of the sequence listing are hereby incorporated by reference.

### BACKGROUND

Pharmaceutical formulations for multiple use are particularly convenient for patients with chronic diseases, in need of frequent treatment.

A pharmaceutical formulation contains a number of excipients in addition to at least one active pharmaceutical ingredient (API). The individual ingredients, excipients as well as API, all serve a specific purpose. The purpose of the tightly controlled pH is to keep the composition stable during its storage and use. The purpose of the surfactant is to stabilise the proteinaceous API and prevent it from adsorbing to containers and production materials. In pharmaceutical formulations for multiple use, the purpose of the antimicrobial preservative is to prevent the growth of bacteria and fungi. Indeed, it is a regulatory requirement that a pharmaceutical formulation for multiple use comprises one or more antimicrobial preservatives, to protect it from microbial growth once the initially sterile container containing it has been penetrated (eg., by a needle), or during its repeated use.

It is well known in the art that mixing a preservative, a surfactant and an antibody is not a straightforward endeavour, nor is pH adjustment of a composition comprising an antibody. When mixed together, undesirable interactions readily occur between the preservative and the surfactant and the preservative and the antibody, resulting in chemical and physical instability. Adjusting the pH can also destabilise the antibody. Such interactions can ruin the quality of the formulation, which then has to be discarded.

For example, it is well known known that producing a pharmaceutical formulation comprising a proteinaceous API, such as an antibody, and one or more antimicrobial preservatives presents a serious challenge (Gupta et Kaisheva (2003) AAPS PharmSci; 5 (2) Article 8; Meyer et al. (2007) Jour Pharm Sci; 96 (12). The critical production step has been observed to be when the solution containing the antimicrobial preservative is mixed with the drug substance comprising the API. Increased formation of API-derived aggregates and particles and even precipitation of the purified, proteinaceous API has been observed to occur. Misfolding of the proteinaceous API is thought to take place due to the exposure of the protein molecule to the hydrophobic surfaces of the antimicrobial preservative.

A common strategy for reducing the formation of aggregates and/or particles is to add a surfactant to the drug product formulation (Randolph T W, Jones L S. 2002. Surfactant-protein interaction in rational design of stable protein formulations: Theory and Practice; pp. 159-175). However, adding surfactant leads to further challenges during the large scale manufacturing of pharmaceutical formulations, as surfactants and antimicrobial preservatives interact when present in higher concentrations.

Therefore, there is a need in the art for a different method of preparing a pharmaceutical formulation that is suitable for multiple use.

### SUMMARY

The current invention relates to a method of formulating a drug substance comprising concizumab into a drug product (pharmaceutical formulation) comprising concizumab, at least one antimicrobial preservative and at least one surfactant.

Disclosed herein is a method of preparing a pharmaceutical formulation of concizumab, comprising the following steps:
obtaining or preparing a drug substance (DS) comprising concizumab as the active pharmaceutical ingredient and having a pH of 5.0-6.5, such as pH 5.5-6.5, preferably pH 6.0;
preparing an aqueous, surfactant-free excipient solution comprising one or two antimicrobial preservatives, selected from the group consisting of benzyl alcohol, chlorobutanol, m-cresol and phenol, and having a pH of 5.0-6.5, such as pH 5.5-6.5, preferably 6.0;
optionally, adjusting the pH of the excipient solution;
mixing the DS and the excipient solution;
adding surfactant to the mixture of the DS and excipient solution;
optionally, adding water.

The purpose of the optional addition of water is to achieve the desired batch volume, according to which all other amounts are calculated in advance. The water added is usually water for injection.

Disclosed herein is the pharmaceutical formulation obtained by the method disclosed herein.

Disclosed herein is the medical use of the pharmaceutical formulation obtained by the method disclosed herein, specifically, its use in the treatment of a coagulopathy.

### Sequence Listing

SEQ ID NO: 1 represents the light chain of concizumab.

SEQ ID NO: 2 represents the heavy chain of concizumab.

### DRAWINGS

FIG. 1 is a flow diagram illustrating manufacturing method 1.

FIG. 2 is a flow diagram illustrating manufacturing method 2.

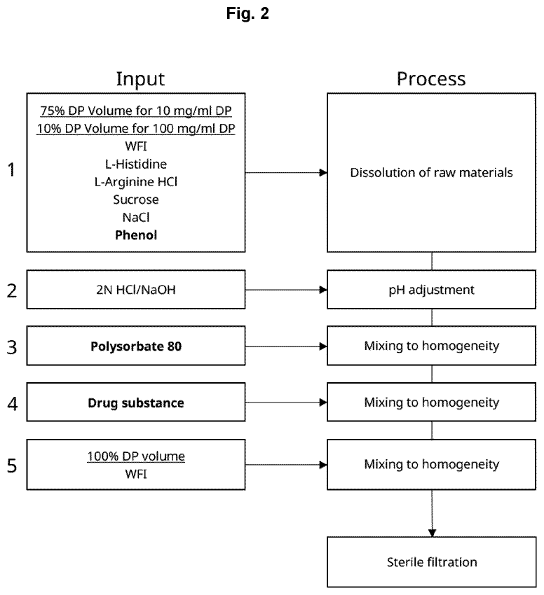

FIG. 3 is a flow diagram illustrating manufacturing method 3.

FIG. 4 is a flow diagram illustrating manufacturing method 4.

FIG. 5 is a flow diagram illustrating manufacturing method 5.

FIG. 6 is a flow diagram illustrating manufacturing method 6.

FIG. 7 is a flow diagram illustrating manufacturing method 7.

### DESCRIPTION

During the manufacture of the pharmaceutical formulation disclosed herein, the means of converting the “drug substance” to the “drug product” (pharmaceutical formulation) was extensively studied, in order to develop a method that allows for the preservative, the surfactant and the antibody to be mixed together, and for the pH to be adjusted, without these ingredients interacting to an unacceptable degree.

The inventors designed a method which allows for compositions comprising the preservative, the surfactant and the antibody to be mixed together, and for the pH to be adjusted, without these ingredients interacting to an unacceptable degree. Furthermore, the method is advantageous because it only requires use of a single tank on the factory floor and because it is particularly easy to implement, in that is easy to calculate the amounts of each composition to be added to the tank.

In the current context, the active pharmaceutical ingredient (API) is a recombinantly produced protein, such as a recombinantly produced antibody, such as a recombinantly produced anti-TFPI antibody, preferably concizumab.

The method disclosed herein may be applied by the pharmaceutical industry, in both pilot production (≥2 L) and commercial production. The method may be applied after upstream and downstream manufacture and purification of the active pharmaceutical ingredient (API) have been completed, i.e., following production of the so-called “drug substance”.

Disclosed herein is a method of preparing a pharmaceutical formulation of concizumab, comprising the following steps:
obtaining or preparing a drug substance (DS) comprising concizumab as the active pharmaceutical ingredient and having a pH of 5.0-6.5, such as pH 5.5-6.5, preferably about pH 6.0;
preparing an aqueous, surfactant-free excipient solution comprising one or two antimicrobial preservatives, selected from the group consisting of benzyl alcohol, chlorobutanol m-cresol and phenol, and having a pH of 5.0-6.5, such as pH 5.5-6.5, preferably 6.0;
optionally, adjusting the pH of the excipient solution;
mixing the DS and the excipient solution;
adding surfactant to the mixture of the DS and excipient solution;
optionally, adding water.

In other words, the method disclosed herein involves dissolving excipients and preservative, but not surfactant, in water, adjusting the pH of the solution to 5.0-6.5, such as 5.5-6.5, preferably about 6.0; mixing the excipient solution with drug substance and then adding surfactant. Finally, water for injection (WFI) may be added to achieve the final volume desired.

Disclosed herein is a method of preparing a pharmaceutical formulation of concizumab, comprising the following steps:
obtaining or preparing a drug substance (DS) comprising concizumab as the active pharmaceutical ingredient (API) and having a pH of 5.0-6.5, such as pH 5.5-6.5, preferably pH 6.0;
preparing an aqueous, surfactant-free excipient solution comprising an antimicrobial preservative selected from the group consisting of benzyl alcohol, chlorobutanol, m-cresol and phenol and having a pH of 5.0-6.5, such as pH 5.5-6.5, preferably about pH 6.0;
optionally, adjusting the pH of the excipient solution;
mixing the DS and the excipient solution;
adding surfactant to the mixture of the DS and excipient solution;
optionally, adding water;
wherein:
the preservative is benzyl alcohol, in a concentration of less than 35 mg/ml in the DS-excipient mixture, and the surfactant subsequently added is Polysorbate 20 or Polysorbate 80; or
the preservative is benzyl alcohol, in a concentration of no more than 35 mg/ml in the DS-excipient mixture, when the surfactant subsequently added is Poloxamer 188; or
the preservative is chlorobutanol, in a concentration of no more than 7.5 mg/ml in the DS-excipient mixture, when the surfactant subsequently added is Polysorbate 20, Polysorbate 80 or Poloxamer 188; or
the preservative is m-cresol, in a concentration of less than 3 mg/ml in the DS-excipient mixture, when the surfactant subsequently added is Polysorbate 20; or
the preservative is m-cresol, in a concentration of no more than 5 mg/ml in the DS-excipient mixture, when the surfactant subsequently added is Polysorbate 80; or
the preservative is m-cresol, in a concentration of less than 10 mg/ml in the DS-excipient mixture, when the surfactant subsequently added is Poloxamer 188; or
the preservative is phenol, in a concentration of less than 6 mg/ml in the DS-excipient mixture, when the surfactant subsequently added is Polysorbate 20; or
the preservative is phenol, in a concentration of less than 8 mg/ml, preferably about 6 mg/ml, in the DS-excipient mixture, when the surfactant subsequently added is Polysorbate 80; or
the preservative is phenol, in a concentration of less than 10 mg/ml in the DS-excipient mixture, when the surfactant subsequently added is Poloxamer 188.

Disclosed herein is a method of preparing a pharmaceutical formulation of concizumab, comprising the following steps:
obtaining or preparing a drug substance (DS) comprising concizumab as the active pharmaceutical ingredient (API) and having a pH of 5.0-6.5, such as pH 5.5-6.5, preferably about pH 6.0;
preparing an aqueous, surfactant-free excipient solution comprising benzyl alcohol and having a pH of 5.0-6.5, such as pH 5.5-6.5, preferably about pH 6.0;
optionally, adjusting the pH of the excipient solution;
mixing the DS and the excipient solution, such that the concentration of benzyl alcohol in the DS-excipient mixture is less than 35 mg/ml;
adding Polysorbate 20 or Polysorbate 80 to the mixture of the DS and excipient solution;
optionally, adding water.

Disclosed herein is a method of preparing a pharmaceutical formulation of concizumab, comprising the following steps:
obtaining or preparing a drug substance (DS) comprising concizumab as the active pharmaceutical ingredient (API) and having a pH of 5.0-6.5, such as pH 5.5-6.5, preferably about pH 6.0;
preparing an aqueous, surfactant-free excipient solution comprising benzyl alcohol and having a pH of 5.0-6.5, such as pH 5.5-6.5, preferably about pH 6.0;
optionally, adjusting the pH of the excipient solution;
mixing the DS and the excipient solution, such that the concentration of benzyl alcohol in the DS-excipient mixture is up to 35 mg/ml;
adding Poloxamer 188 to the mixture of the DS and excipient solution;
optionally, adding water.

Disclosed herein is a method of preparing a pharmaceutical formulation of concizumab, comprising the following steps:
obtaining or preparing a drug substance (DS) comprising concizumab as the active pharmaceutical ingredient (API) and having a pH of 5.0-6.5, such as pH 5.5-6.5, preferably about pH 6.0;
preparing an aqueous, surfactant-free excipient solution comprising chlorobutanol and having a pH of 5.0-6.5, such as pH 5.5-6.5, preferably about pH 6.0;
optionally, adjusting the pH of the excipient solution;
mixing the DS and the excipient solution, such that the concentration of chlorobutanol in the DS-excipient mixture is up to 7.5 mg/ml;
adding Polysorbate 20, Polysorbate 80 or Poloxamer 188 to the mixture of the DS and excipient solution;
optionally, adding water.

Disclosed herein is a method of preparing a pharmaceutical formulation of concizumab, comprising the following steps:
obtaining or preparing a drug substance (DS) comprising concizumab as the active pharmaceutical ingredient (API) and having a pH of 5.0-6.5, such as pH 5.5-6.5, preferably about pH 6.0;
preparing an aqueous, surfactant-free excipient solution comprising m-cresol and having a pH of 5.0-6.5, such as pH 5.5-6.5, preferably about pH 6.0;
mixing the DS and the excipient solution, such that the concentration of m-cresol in the DS-excipient mixture is less than 3 mg/ml;
adding Polysorbate 20 to the mixture of the DS and excipient solution;
optionally, adding water.

Disclosed herein is a method of preparing a pharmaceutical formulation of concizumab, comprising the following steps:
obtaining or preparing a drug substance (DS) comprising concizumab as the active pharmaceutical ingredient (API) and having a pH of 5.0-6.5, such as pH 5.5-6.5, preferably about pH 6.0;
preparing an aqueous, surfactant-free excipient solution comprising m-cresol and having a pH of 5.0-6.5, such as pH 5.5-6.5, preferably about pH 6.0;
optionally, adjusting the pH of the excipient solution;
mixing the DS and the excipient solution, such that the concentration of m-cresol in the DS-excipient mixture is no greater than 5 mg/ml;
adding Polysorbate 80 to the mixture of the DS and excipient solution;
optionally, adding water.

Disclosed herein is a method of preparing a pharmaceutical formulation of concizumab, comprising the following steps:
obtaining or preparing a drug substance (DS) comprising concizumab as the active pharmaceutical ingredient (API) and having a pH of 5.0-6.5, such as pH 5.5-6.5, preferably about pH 6.0;
preparing an aqueous, surfactant-free excipient solution comprising m-cresol and having a pH of 5.0-6.5, such as pH 5.5-6.5, preferably about pH 6.0;
optionally, adjusting the pH of the excipient solution;
mixing the DS and the excipient solution, such that the concentration of m-cresol in the DS-excipient mixture is less than 10 mg/ml;
adding Poloxamer 188 to the mixture of the DS and excipient solution;
optionally, adding water.

Disclosed herein is a method of preparing a pharmaceutical formulation of concizumab, comprising the following steps:
obtaining or preparing a drug substance (DS) comprising concizumab as the active pharmaceutical ingredient (API) and having a pH of 5.0-6.5, such as pH 5.5-6.5, preferably about pH 6.0;
preparing an aqueous, surfactant-free excipient solution comprising phenol and having a pH of 5.0-6.5, such as pH 5.5-6.5, preferably about pH 6.0;
optionally, adjusting the pH of the excipient solution;
mixing the DS and the excipient solution, such that the concentration of phenol in the DS-excipient mixture is less than 6 mg/ml;
adding Polysorbate 20 to the mixture of the DS and excipient solution;
optionally, adding water.

Disclosed herein is a method of preparing a pharmaceutical formulation of concizumab, comprising the following steps:
obtaining or preparing a drug substance (DS) comprising concizumab as the active pharmaceutical ingredient (API) and having a pH of 5.0-6.5, such as pH 5.5-6.5, preferably about pH 6.0;
preparing an aqueous, surfactant-free excipient solution comprising phenol and having a pH of 5.0-6.5, such as pH 5.5-6.5, preferably about pH 6.0;
optionally, adjusting the pH of the excipient solution;
mixing the DS and the excipient solution, such that the concentration of phenol in the DS-excipient mixture is less than 8 mg/ml, such as 5.5-6.5 mg/ml, preferably 6 mg/ml;
adding Polysorbate 80 to the mixture of the DS and excipient solution;
optionally, adding water.

Disclosed herein is a method of preparing a pharmaceutical formulation of concizumab, comprising the following steps:
obtaining or preparing a drug substance (DS) comprising concizumab as the active pharmaceutical ingredient (API) and having a pH of 5.0-6.5, such as pH 5.5-6.5, preferably about pH 6.0;
preparing an aqueous, surfactant-free excipient solution comprising less than 55 mg/ml, such as no more than 45 mg/ml phenol and having a pH of 5.0-6.5, such as pH 5.5-6.5, preferably about pH 6.0;
optionally, adjusting the pH of the excipient solution;
mixing the DS and the excipient solution, such that the concentration of phenol in the DS-excipient mixture is less than 8 mg/ml, such as 5.5-6.5 mg/ml, preferably 6 mg/ml;
adding Polysorbate 80 to the mixture of the DS and excipient solution;
optionally, adding water.

Disclosed herein is a method of preparing a pharmaceutical formulation of concizumab, comprising the following steps:
obtaining or preparing a drug substance (DS) comprising concizumab as the active pharmaceutical ingredient (API) and having a pH of 5.0-6.5, such as 5.5-6.5, preferably about 6.0; preparing an aqueous, surfactant-free excipient solution comprising L-arginine HCl, L-histidine, sucrose, sodium chloride, phenol and water, such that the concentration of phenol is less than 55 mg/ml, such as no greater than 45 mg/ml, and adjusting the pH to 5.0-6.5, such as 5.5-6.5, preferably about 6.0;
optionally, adjusting the pH of the excipient solution;
adding DS to the excipient solution, such that the concentration of phenol in the mixture is less than 8 mg/ml, such as about 5.5-6.5, preferably about 6 mg/ml;
adding polysorbate 80 to the mixture to a final concentration of 0.1-0.3 mg/ml, preferably about 0.25 mg/ml;
optionally, adding WFI to the final batch volume.

Disclosed herein is a method of preparing a pharmaceutical formulation of concizumab, comprising the following steps:
obtaining or preparing a drug substance (DS) comprising concizumab as the active pharmaceutical ingredient (API) and having a pH of 5.0-6.5, such as pH 5.5-6.5, preferably about pH 6.0;
preparing an aqueous, surfactant-free excipient solution comprising phenol and having a pH of 5.0-6.5, such as pH 5.5-6.5, preferably about pH 6.0;
optionally, adjusting the pH of the excipient solution;
mixing the DS and the excipient solution, such that the concentration of phenol in the DS-excipient mixture is less than 10 mg/ml;
adding Poloxamer 188 to the mixture of the DS and excipient solution;
optionally, adding water.

Unless otherwise explicitly mentioned, the conditions specified herein apply when the method of preparing the pharmaceutical formulation is conducted at room temperature.

In the context of the current invention, concizumab is the active pharmaceutical ingredient (API). Concizumab is described in WO2010/072691, the content of which is hereby incorporated by reference in its entirety. In WO2010/072691, concizumab is referred to as “HzTFPI4F36” and “mAbTFPI2021” and the polypeptide sequences of its light and heavy chains are provided in SEQ ID NO: 21 and SEQ ID NO: 24, respectively. For the avoidance of doubt, the polypeptide sequences of the light and heavy chains of concizumab are also provided herein as SEQ ID NO:1 and SEQ ID NO: 2, respectively.

Expression of concizumab in suitable cell lines is also described in WO2010/072691.

In order to prepare a drug substance comprising concizumab, upstream and downstream manufacturing processes are performed.

The term “upstream manufacturing process” refers to the process of manufacturing concizumab from a stable cell bank that is capable of expressing it. Examples of cells that are capable of recombinantly expressing proteinaceous active pharmaceutical ingredients are well known in the art and include mammalian HEK293, CHO, BHK, NSO and human retina cells.

The term “unprocessed bulk” refers to the product of an upstream manufacturing process. In the context of the present invention, unprocessed bulk contains recombinantly expressed concizumab, cellular debris and components from the medium/media used to nourish the cells expressing concizumab.

Unprocessed bulk is typically filtered or centrifuged and then subject to a downstream purification process which comprises or consists of several purification methods. Purification methods for monoclonal antibodies, such as concizumab, are well known in the art and are described, for example, in Pete Gagnon: Purification Tools for Monoclonal Antibodies (1996) ISBN-9653515-9-9. Such methods include affinity chromatography (such as protein A chromatography), ion exchange chromatography (such as anion exchange chromatography and cation exchange chromatography), size exclusion chromatography, hydrophobic interaction chromatography, ultrafiltration and virus filtration. One purification process that can be applied in the context of the current invention is that described in WO2009/138484, the content of which is hereby incorporated by reference.

The term “drug substance” herein refers to the final output of the downstream purification process. “Drug substance” is well known in the art as referring to “any [substance or] mixture of substances intended to be used in the manufacture of a drug (medicinal) product and that, when used in the production of a drug, becomes an active ingredient of the drug product” (see, for example, EMA ICH Q7a: “Good manufacturing practice for active pharmaceutical ingredients”). It is well known to the person skilled in the art that a drug substance must be essentially free of impurities, to the extent required by regulatory authorities. Impurities that are removed from the unprocessed bulk and semi-processed bulk during the downstream purification process include: media components from the upstream manufacturing process, cellular debris (such as host cell protein and DNA), any impurity derived from downstream process steps (such as protein A from a protein A chromatography column), microorganisms (such as vira) and aggregates or irreversibly damaged forms of concizumab, such as high molecular weight protein (HMWP).

In the context of the present invention, “concizumab drug substance”, contains concizumab as the active pharmaceutical ingredient and excipients such as the buffer used during downstream concizumab purification. The concizumab drug substance is free—or substantially free, to the levels required by regulatory authorities—of the following: media components from the upstream manufacturing process, microorganisms (such as vira), cellular debris (such as host cell protein and DNA), any impurity derived from any downstream process step (such as protein A from a protein A chromatography column) and aggregates or irreversibly damaged forms of concizumab, such as high molecular weight protein (HMWP).

Concizumab drug substance may be prepared using the downstream purification process described in WO2009/138484.

The drug substance does not contain an antimicrobial preservative.

When the drug substance contains an antibody such as concizumab as the active pharmaceutical ingredient, the former is typically in the form of a liquid composition. In this case, the drug substance may comprise 80-300 mg/ml concizumab, preferably 100-140 mg/ml concizumab.

Drug substances containing proteins may also be freeze-dried or spray-dried solids. However, it is not usually necessary to freeze-dry or spray-dry antibodies.

The drug substance comprising concizumab may further comprise a buffer, a tonicity agent and salts.

The drug substance comprising concizumab may further comprise a buffer, a tonicity agent, a viscosity lowering agent and a stabiliser.

The drug substance comprising concizumab may further comprise histidine as a buffer, sucrose as a tonicity agent, sodium chloride as a viscosity lowering agent and arginine or arginine hydrochloride as a stabiliser.

In one preferred embodiment, the drug substance comprises 120 mg/ml concizumab in a diafiltration buffer containing 33 mmol/L histidine, 25 mmol/L arginine, 25 mmol/L NaCl, 150 mmol/L sucrose and 0.01 mg/mL polysorbate 80, pH 6.0.

Production of a drug substance such as “drug substance comprising concizumab” precedes implementation of the formulation method disclosed herein, whose aim is to create the final “drug product” or pharmaceutical formulation.

In the context of the present invention, the term “drug product” is synonymous with the term “pharmaceutical formulation”. In the context of the present invention, the term “drug product comprising concizumab” is synonymous with the term “pharmaceutical formulation comprising concizumab”.

In order to implement the current invention, “an aqueous, surfactant-free excipient solution comprising at least one antimicrobial preservative”, must be prepared. Unless otherwise precluded by context, the terms “aqueous, surfactant-free excipient solution comprising at least one antimicrobial preservative”, “aqueous, surfactant-free excipient solution”, “surfactant-free excipient solution” and “excipient solution” are herein used synonymously.

The use of excipients in pharmaceutical compositions is well-known to the skilled person. For convenience, reference is made to Remington: The Science and Practice of Pharmacy, 20th edition, 2000.

The excipient solution may comprise the same buffer, tonicity agent, viscosity-lowering agent and stabiliser as the drug substance. The excipient solution may comprise the same buffer, tonicity agent and salt or salts as the drug substance. However, the concentrations of the excipients in the drug substance and the excipient solution may be different.

The excipient solution must comprise at least one antimicrobial preservative or “preservative”. The excipient solution may comprise one or two preservatives. The preservative or combination of preservatives must be compatible with the route of administration, be effective against microorganisms, including fungi and bacteria, and must not be toxic to human beings at the concentrations required to be effective against microorganisms.

The concentration of the antimicrobial preservative in the pharmaceutical formulation must be within the range defined in the relevant pharmacopoeia, such as in the European Pharmacopoeia (Pharmacopoeia Europaea, Ph. Eur.) if regulatory approval is sought in Europe or the United States Pharmacopeia (USP) pharmacopoeia if regulatory approval is sought in the United States. Such pharmacopoeia define the minimum concentration(s) of antimicrobial preservatives required to achieve the bacteriostatic effect specified by the respective regulatory authorities for multiple dose pharmaceutical formulations. To limit any toxic effect of the antimicrobial preservative in question, it is the minimum concentration that must be present in the drug product, as is well known to the person skilled in the art.

The concentration of the antimicrobial preservative in the excipient solution, must be adjusted such as to achieve this without causing any of the other ingredients to become unstable.

In one embodiment, the excipient solution comprises one or two antimicrobial preservatives, histidine, sucrose, arginine hydrochloride and sodium chloride.

In the context of the present invention, the at least one antimicrobial preservative may be selected from the group consisting of benzyl alcohol, chlorobutanol, m-cresol, methylparaben, phenol and propylparaben, or a combination thereof.

Two antimicrobial preservatives may be selected from the group consisting of benzyl alcohol, chlorobutanol, m-cresol, methylparaben, phenol and propylparaben.

Two antimicrobial preservatives may be selected from the group consisting of benzyl alcohol, chlorobutanol, m-cresol and phenol.

A single antimicrobial preservative may be selected from the group consisting of benzyl alcohol, chlorobutanol, m-cresol, methylparaben, phenol and propylparaben.

A single antimicrobial preservative may be selected from the group consisting of benzyl alcohol, chlorobutanol, m-cresol and phenol.

The single antimicrobial preservative may be benzyl alcohol. In one embodiment, the concentration of benzyl alcohol in the mixture of the drug substance and the excipient solution is 35 mg/ml or less. In one embodiment, the concentration of benzyl alcohol in the mixture of the drug substance and the excipient solution is no greater than 20 mg/ml. The concentration of benzyl alcohol in the pharmaceutical formulation may contain about 9-11 mg/ml benzyl alcohol, such as about 10 mg/ml benzyl alcohol.

The single antimicrobial preservative may be chlorobutanol. In one embodiment, the concentration of chlorobutanol in the mixture of the drug substance and the excipient solution is 7.5 mg/ml or less. In one embodiment, the concentration of chlorobutanol in the mixture of the drug substance and the excipient solution is 6.5 mg/ml or less. The pharmaceutical formulation of concizumab may contain about 3.0 to 7.5 mg/ml chlorobutanol, such as about 5 mg/ml chlorobutanol.

The single antimicrobial preservative may be m-cresol. In one embodiment, the concentration of the m-cresol in the mixture of the drug substance and the excipient solution is less than 10 mg/ml. In one embodiment, the concentration of the m-cresol in the mixture of the drug substance and the excipient solution is no greater than 5 mg/ml. In one embodiment, the concentration of the m-cresol in the mixture of the drug substance and the excipient solution is 3 mg/ml or less. In one embodiment, the concentration of the m-cresol in the mixture of the drug substance and the excipient solution is less than 3 mg/ml. The pharmaceutical formulation of concizumab may contain about 2.7-3.2 mg/ml m-cresol, such as about 3 mg/ml m-cresol.

In a preferred embodiment, the single antimicrobial preservative is phenol. In one embodiment, the concentration of phenol in the excipient solution is less than 55 mg/ml, such as no greater than about 45 mg/ml. The concentration of phenol in the excipient solution may be about 4-45 mg/ml, such as 6-45 mg/ml, such as about 4-40 mg/ml, such as about 4-10 mg/ml, such as about 11-20 mg/ml, such as about 11-15 mg/ml, such as about 16-20 mg/ml, such as about 21-30 mg/ml, such as about 21-25 mg/ml, such as about 26-30 mg/ml, such as about 31-40 mg/ml, such as about 31-35 mg/ml, such as about 36-40 mg/ml, such as about 41-45 mg/ml.

In one embodiment, the concentration of phenol in the mixture of the drug substance and the excipient solution is at least 2.5 mg/ml, such as at least 3.0 mg/ml, and less than 10 mg/ml, such as less than about 8 mg/ml; such as about 2.5-5.0 mg/ml; such as about 3.0-5.0 mg/ml; such as about 3.0-4.0 mg/ml; such as about 3.0 mg/ml, such as least about 3.2 mg/ml, such as about 3.5 mg/ml, such as about 4.0-7.5 mg/ml, such as about 4.0-5.5 mg/ml, such as about 4.0-5.0 mg/ml, such as about 4.5-5.5 mg/ml, such as about 5.5-7.5, such as about 5.5-6.5, such as about about 6 mg/ml; such as no greater than about 6 mg/ml.

The concentration of phenol in the pharmaceutical formulation may be 3-6 mg/ml, preferably 3-4 mg/ml, even more preferably about 3.5 mg/ml.

The drug substance comprising concizumab may comprise a buffer. The excipient solution may comprise a buffer. The mixture of the drug substance and the excipient solution may comprise a buffer. The pharmaceutical formulation comprising concizumab may comprise a buffer.

The pH at which a protein's positive charge balances its negative charge is its isoelectric point (ph. At pH values above their pls, proteins become progressively electronegative. Below their pls, they become progressively electropositive. In the context of the current invention, the pH of the buffer may be within the pl range of concizumab (6.2-6.7), such that concizumab has a neutral net charge. In one embodiment, the buffer has a pKa value ±1 pH unit from the target pH of the composition.

In one embodiment, the buffer is a suitable pharmaceutically acceptable buffer, which comprises both a pharmaceutically acceptable base and a pharmaceutically acceptable acid. In some embodiments, the buffer may be a salt. In one embodiment, the buffer has a pKa of between 4 and 8, such as between 5 and 7.

Pharmaceutically acceptable acids and bases may be inorganic or organic, non-toxic acids/bases well-known in the art. Examples of pharmaceutically acceptable acids and bases are disodium acetate, sodium carbonate, citrate, histidine, lysine, arginine, maleate, succinate, sodium dihydrogen phosphate, disodium hydrogen phosphate and sodium phosphate, or mixtures thereof. Each one of these specific buffers constitutes an alternative embodiment of the invention. In one embodiment, the buffer is histidine, maleate, succinate or phosphate. In one embodiment, the buffer is histidine, preferably L-histidine.

In one embodiment, the composition is buffered to a pH of between 5 and 7, such as a pH of 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9 or 7.0, or to a pH as defined by any ranges in-between. In one embodiment, the composition is buffered to a pH of between 5.0 and 6.5. In one embodiment, the composition is buffered to a pH of between 5.5 and 6.5. In a preferred embodiment, the composition is buffered to a pH of about 6.0.

To adjust pH, hydrochloric acid (HCl) or sodium hydroxide (NaOH) may be added to the excipient solution.

The drug substance comprising concizumab may comprise a stabiliser. The aqueous, surfactant-free excipient solution comprising at least one antimicrobial preservative may further comprise a stabiliser. The mixture of the drug substance and the excipient solution may further comprise a stabiliser. The pharmaceutical formulation comprising concizumab may further comprise a stabiliser.

The drug substance comprising concizumab may further comprise a viscosity-lowering agent. The aqueous, surfactant-free excipient solution comprising at least one antimicrobial preservative may further comprise a viscosity-lowering agent. The mixture of the drug substance and the excipient solution may further comprise a viscosity-lowering agent. The pharmaceutical formulation comprising concizumab may further comprise a viscosity-lowering agent. In one embodiment, the viscosity-lowering agent is selected from the group consisting of HisHCI, LysHCI, ArgHCI, NaGlu, NaCl, NaAc, Na2SO4, and NH4Cl, imidazole, camphorsulfonic acid, Dipicolinic acid salts, scopolamine, 1-(3-aminopropyl)-2-methyl-1H-imidazole, procaine, lidocaine, chloroquine, 4-aminopyridine, 1-butyl-3-methylimidazolium and 4-(3-butyl-1-imidazolio)-1-butanesulfonate, or a combination thereof. In a preferred embodiment, the viscosity-lowering agent is arginine hydrochloride (ArgHCI). In another preferred embodiment, the viscosity-lowering agent is sodium chloride (NaCl). In another preferred embodiment, the viscosity-lowering agent is a combination of arginine hydrochloride and sodium chloride.

The drug substance comprising concizumab may further comprise a tonicity modifying agent. The aqueous, surfactant-free excipient solution comprising at least one antimicrobial preservative may further comprise a tonicity modifying agent. The mixture of the drug substance and the excipient solution may further comprise a tonicity modifying agent. The pharmaceutical formulation comprising concizumab may further comprise a tonicity modifying agent.

Examples of suitable tonicity modifying agents include salts (eg. sodium chloride), polyhydric alcohols (eg. propyleneglycol, glycerol, xyllitol. mannitol or D-sorbitol), monosaccarides (glucose or maltose), disaccarides (eg. sucrose), amino acids (eg. L-glycine, L-histidine, arginine, lysine, isoleucine, aspartic acid, tryptophane, threonine) or mixtures thereof. In one embodiment, the tonicity modifying agent is sucrose, mannitol or propylene glycol. In a preferred embodiment, the tonicity modifying agent is sucrose.

In one embodiment, the buffer and/or salt (as described above) also acts as a tonicity modifier, or the tonicity modifier also acts as a buffer and/or salt; in which case the concentration of the tonicity modifier will be calculated accordingly.

In one embodiment, the amount of tonicity modifying agent in the pharmaceutical composition of concizumab is between 50 and 250 mM, such as between 100 and 200 mM, for example any one of 100, 110, 120, 130, 140, 150, 160, 170, 180, 190 or 200, or any range in between.

In one preferred embodiment, the pharmaceutical composition of concizumab, comprises about 150 mM sucrose.

In one preferred embodiment, the pharmaceutical composition of concizumab is isotonic.

The drug substance comprising concizumab may further comprise one or more salts. The excipient solution may comprise one or more salts. The mixture of the drug substance and the excipient solution may further comprise one or more salts. The pharmaceutical formulation comprising concizumab may further comprise one or more salts. The salt or salts may have more than one function, as is well known to the person skilled in the art. For example: the salt may have a buffering capacity at the relevant pH; the salt may lower viscosity; the salt may be a stabiliser.

In one embodiment, the salt is an inorganic salt. In one embodiment, the inorganic salt is sodium chloride or magnesium chloride.

In one embodiment, the salt is an organic salt. In one embodiment, the salt is that of an amino acid. In one embodiment the salt is that of the L-stereoisomer of an amino acid. In one embodiment, the salt is that of arginine, glycine, lysine, aspartic acid or glutamic acid, or a combination thereof. In one embodiment, the amino acid is arginine or glycine. In one embodiment, the amino acid is arginine, such as L-arginine. The amino acid can be added to the composition in its salt form or in its free form.

In one preferred embodiment, the organic salt is arginine hydrochloride.

In one embodiment, the salts are a combination of an inorganic and an organic salt.

In one embodiment, the salts are sodium chloride, magnesium chloride, sodium thiocyanate, ammonium thiocyanate, ammonium sulfate, ammonium chloride, calcium chloride, arginine hydrochloride, zinc chloride, sodium acetate, one or more amino acids, or a combination of two or more of these.

In a preferred embodiment, the salts are sodium chloride and arginine hydrochloride.

The use of a surfactant in pharmaceutical compositions is well-known to the skilled person. For convenience, reference is made to Remington: The Science and Practice of Pharmacy, 20^{th} edition, 2000.

In the method disclosed herein, a surfactant is added to the mixture of the drug substance and excipient solution. Thus, the pharmaceutical formulation comprising concizumab comprises a surfactant.

In one embodiment, the surfactant is selected from a polysorbate or polyoxypropylene-polyoxyethylene block polymers (eg. a poloxamer such as Pluronic® F68, poloxamer 188 and 407, Triton X-100) or a polyoxyethylene or polyethylene derivative, such as an alkylated or alkoxylated derivative (Polysorbates, e.g. Tween-20, Tween-40, Tween-80 and Brij-35).

Preferably, the surfactant is selected from the group consisting of polysorbate 20, polysorbate 80 or polyoxamer 188. The surfactant may be polysorbate 20. The surfactant may be polysorbate 80. The surfactant may be poloxamer 188.

There is no surfactant in the excipient solution. However, there may be some surfactant in the concizumab drug substance. For example, the drug substance may contain 0.0-0.1 polysorbate 80, such as 0.0-0.02 mg/ml polysorbate 80, preferably about 0.01 polysorbate 80.

The pharmaceutical formulation may comprise 0.1-2.0 mg/ml, such as 0.1-0.3 mg/ml, such as 0.1-0.2 mg/ml, such as 0.2-0.3 mg/ml, such as about 0.25 mg/ml polysorbate 20.

The pharmaceutical formulation may comprise 0.1-2.0 mg/ml, such as 0.1-0.3 mg/ml, such as 0.1-0.2 mg/ml, such as 0.2-0.3 mg/ml, such as about 0.25 mg/ml polysorbate 80.

The pharmaceutical formulation may comprise 0.1-10 mg/ml, such as 0.1-2.0 mg/ml, such as 0.9-1.1 poloxamer 188, such as about 1.0 mg/ml poloxamer 188.

Any water added to the drug substance, the excipient solution, the mixture of the drug substance and the excipient solution or the mixture of the drug substance, excipient solution and surfactant will typically be water for injection (WFI).

The pharmaceutical formulation obtained by the method disclosed herein is chemically and physically stable, being essentially free of impurities. The pharmaceutical formulation is a clear to slightly opalescent and colourless to slightly yellow liquid, essentially free from visible particles.

The pharmaceutical formulation disclosed herein may comprise 5-110 mg/ml, such as 5-15 mg/ml, such as about 10 mg/ml, such as 15-25 mg/ml, such as about 20 mg/ml, such as 25-35 mg/ml, such as about 30 mg/ml, such as 35-45 mg/ml, such as about 40 mg/ml, such as 45-55 mg/ml, such as about 50 mg/ml, such as 55-65 mg/ml, such as about 60 mg/ml, such as 65-75 mg/ml, such as about 70 mg/ml, such as 75-85 mg/ml, such as about 80 mg/ml, such as 85-95 mg/ml, such as about 90 mg/ml, such as 95-105 mg/ml, such as about 100 mg/ml concizumab.

The pharmaceutical formulation disclosed herein preferably comprises about 10 mg/ml, about 40 mg/ml or about 100 mg/ml concizumab.

The pharmaceutical formulation disclosed herein preferably comprises concizumab, histidine, sucrose, sodium chloride, arginine hydrochloride, polysorbate 80 and having a pH of 5.5-6.5, preferably about 6.0.

In one preferred embodiment, the pharmaceutical formulation consists of:
Concizumab: about 100 mg/ml
Phenol: about 0.35% (3.5 mg/ml)
L-Histidine: about 33 mM (5.12 mg/ml)
Sodium chloride: about 25 mM (1.46 mg/ml)
L-Arginine HCl: about 25 mM (5.27 mg/ml)
Sucrose: about 150 mM (51.3 mg/ml)
Polysorbate 80: about 0.25 mg/ml
Water
pH 6.0

In another preferred embodiment, the pharmaceutical formulation consists of:
Concizumab: about 40 mg/ml
Phenol: about 0.35% (3.5 mg/ml)
L-Histidine: about 33 mM (5.12 mg/ml)
Sodium chloride: about 25 mM (1.46 mg/ml)
L-Arginine HCl: about 25 mM (5.27 mg/ml)
Sucrose: about 150 mM (51.3 mg/ml)
Polysorbate 80: about 0.25 mg/ml
Water
pH 6.0

In a third preferred embodiment, the pharmaceutical formulation consists of:
Concizumab: about 10 mg/ml
Phenol: about 0.35% (3.5 mg/ml)
L-Histidine: about 33 mM (5.12 mg/ml)
Sodium chloride: about 25 mM (1.46 mg/ml)
L-Arginine HCl: about 25 mM (5.27 mg/ml)
Sucrose: about 150 mM (51.3 mg/ml)
Polysorbate 80: about 0.25 mg/ml
Water
pH 6.0

### Therapeutic Applications

The pharmaceutical formulation disclosed herein, obtained by means of the method disclosed and claimed herein, which in turn is embodied by manufacturing method 1, may be used for the treatment of subjects in need thereof. Such subjects typically have at least one coagulopathy.

As used herein, the term “subject” includes any human or non-human animal; preferably a human subject.

The human subject may be an adult. The human subject may be a child. The human subject may be an infant. The human subject may be a 0- to 12-year old child. The human subject may be a 12- to 18-year old child.

The term “non-human animal” includes all other vertebrates, including mammals such as non-human primates, domestic animals and laboratory animals such as rabbits, rats and mice.

The term “coagulopathy”, as used herein, refers to an increased haemorrhagic tendency which may be caused by any qualitative or quantitative deficiency of any pro-coagulative component of the normal coagulation cascade, or any upregulation of fibrinolysis. A coagulopathy may be congenital and/or acquired and/or iatrogenic and is identified by a person skilled in the art.

The pharmaceutical formulation disclosed herein may be used for the treatment of a congenital coagulopathy. Congenital coagulopathies have a genetic etiology.

The pharmaceutical formulation disclosed herein may be used for the treatment of a congenital coagulopathy selected from the group consisting of Factor V deficiency, East Texas bleeding disorder, Factor VII deficiency, haemophilia A (Factor VIII deficiency), haemophilia B (Factor IX deficiency), Factor XI deficiency, von Willebrand's disease; certain platelet disorders such as Glanzmann's thrombasthenia and Bernard-Soulier syndrome; and certain connective tissue disorders, such as Ehlers-Danlos Syndrome.

The pharmaceutical formulation disclosed herein may be used for the treatment of a congenital coagulopathy selected from the group consisting of haemophilia A (Factor VIII deficiency) and haemophilia B (Factor IX deficiency).

The pharmaceutical formulation disclosed herein may be used for the treatment of an acquired coagulopathy. Acquired coagulopathies typically result from or are manifestations of other diseases or disease states, including: infections such as HIV; certain types of cancer, such as leukemia; renal disease, such as hemolytic uremic syndrome; liver disease such as hepatitis; autoimmune disease, such as systemic lupus erythematosus or acquired thrombotic thrombocytopenic purpura; acute traumatic coagulopathy and/or disseminated intravascular coagulopathy (DIC).

The pharmaceutical formulation disclosed herein may be used for the treatment of an acquired deficiency of one or more coagulation factors. The pharmaceutical formulation disclosed herein may be used for the treatment of an acquired deficiency of one or more serine proteases. The pharmaceutical formulation disclosed herein may be used for the treatment of acquired thrombocytopaenia. The pharmaceutical formulation disclosed herein may be used for the treatment of an acquired coagulopathy selected from the group consisting of acquired haemophilia A (FVIII deficiency) and acquired haemophilia B (FIX deficiency).

The pharmaceutical formulation disclosed herein may be used for the treatment of a coagulopathy which is partly congenital and partly acquired. Haemophilia A with “inhibitors” (that is, allo-antibodies against factor VIII) and haemophilia B with “inhibitors” (that is, allo-antibodies against factor IX) are non-limiting examples of coagulopathies that are partly congenital and partly acquired.

The pharmaceutical formulation disclosed herein may be used for the treatment of an iatrogenic coagulopathy. Iatrogenic coagulopathies are caused by the toxic effects of prescription drugs or other medical therapy. One non-limiting example of an iatrogenic coagulopathy is serine protease deficiency caused by vitamin K deficiency, which is in turn caused by administration of a vitamin K antagonist or platelet aggregation inhibitor, such as heparin, aspirin or warfarin, that may initially have been prescribed to treat thromboembolic disease. A second, non-limiting example of an iatrogenic coagulopathy is the dilution of coagulation factors and platelets that results from excessive and/or inappropriate fluid therapy. The pharmaceutical formulation disclosed herein may be used for the treatment of an iatrogenic serine protease deficiency and/or thrombocytopaenia.

In one preferred embodiment, the coagulopathy is haemophilia A. In another preferred embodiment, the coagulopathy is haemophilia B. In another preferred embodiment, the coagulopathy is haemophilia A with inhibitors. In another preferred embodiment, the coagulopathy is haemophilia B with inhibitors. In another preferred embodiment, the coagulopathy is caused by thrombocytopenia. In another preferred embodiment, the coagulopathy is von Willebrand's disease. In another embodiment, the coagulopathy is a platelet disorder such as Glanzmann's thrombasthenia. In another embodiment, the coagulopathy is a platelet disorder such as Bernard-Soulier syndrome. In another embodiment, the coagulopathy is a connective tissue disorder such as Ehlers-Danlos Syndrome. In another embodiment, the coagulopathy is a coagulation factor deficiency. In another embodiment, the coagulopathy is a serine protease deficiency.

The pharmaceutical formulation disclosed herein may be used to prevent or treat haemorrhage (bleeding episodes). In one embodiment, the haemorrhage is associated with a coagulation factor deficiency, such as a serine protease deficiency. In one embodiment, the haemorrhage is associated with a FII, FV, FVII, FVIII, FIX, FX FXI and/or FXIII deficiency. In one embodiment, the haemorrhage is associated with trauma. In another embodiment, haemorrhage is associated with surgery. In another embodiment, haemorrhage is associated with haemorrhagic gastritis and/or enteritis. In another embodiment, haemorrhage is associated with an ulcer in the gastrointestinal canal, such as a gastric ulcer. In another embodiment, the haemorrhage is profuse uterine bleeding, such as in connection with abortion, placental abruption, postpartum haemorrhage (PPH) or menorrhagia. In another embodiment, haemorrhage occurs in organs with a limited possibility for mechanical haemostasis, such as intracranially (ICH), intraaurally, intraocularly or intrauterine. In another embodiment, the haemorrhage is associated with anticoagulant therapy.

The pharmaceutical formulation disclosed herein may be used to treat diseases that may ensue from a congenital and/or acquired and/or iatrogenic coagulopathy but have not been diagnosed as such. For example, the pharmaceutical formulation disclosed herein may be used for the treatment of menorrhagia, wherein underlying causes may be bleeding disorders such as a vWF deficiency and/or endometriosis and/or cancer.

Use of the pharmaceutical formulation disclosed herein may significantly reduce blood loss and/or significantly reduce the annual bleeding rate in subjects suffering from any one or more of the above diseases or disease manifestations.

Use of the pharmaceutical composition disclosed herein may reduce clotting time without causing transient thrombocytopaenia.

The term “treatment”, as used herein, refers to the medical therapy of any human or other animal subject in need thereof. Said subject is expected to have undergone physical examination by a medical practitioner or a veterinary medical practitioner, who has given a tentative or definitive diagnosis which would indicate that the use of said specific treatment is beneficial to the health of said human, or other, subject. The timing and purpose of said treatment may vary from one individual to another, according to the status quo of the subject's health. Thus, said treatment may be prophylactic, palliative and/or symptomatic. In terms of the present invention, prophylactic, palliative and/or symptomatic treatments may represent separate aspects of the invention.

The pharmaceutical formulation disclosed herein is preferably administered prophylactically. The pharmaceutical formulation disclosed herein may be administered therapeutically (on demand).

The pharmaceutical formulation is administered to a subject suffering from a coagulopathy as described above, in an amount sufficient to alleviate or partially arrest the condition or one or more of its symptoms. Such therapeutic treatment may result in a decrease in severity of disease symptoms, or an increase in frequency or duration of symptom-free periods. An amount adequate to accomplish this is defined as a “therapeutically effective amount”. For example, where the treatment is in response to haemorrhage or an expectation thereof, therapy may result in a decrease in the volume of blood lost or in the annual bleeding rate.

In prophylactic or preventative applications, the pharmaceutical composition is administered to a subject in an amount sufficient to prevent or reduce the subsequent effects of the condition, or one or more of its symptoms. An amount adequate to accomplish this is defined as a “prophylactically effective amount”. For example, where the treatment is to prevent unwanted bleeding, a prophylactic effect may be defined as the prevention of bleeding or a reduced period or quantity of bleeding compared to that that would be seen in the absence of the modulator.

Effective amounts for each purpose will depend on the severity of the disease or injury as well as the weight and general state of the subject.

Prophylactic use of the pharmaceutical composition disclosed herein may reduce the annual bleeding rate (ABR) of a subject with a coagulopathy. In this context, “annual bleeding rate” refers to spontaneous bleeds, not traumatic bleeds or those related to surgery. In one embodiment, prophylactic use of the pharmaceutical composition disclosed herein may result in the ABR of a subject with coagulopathy being less than it was or would be when the subject received or receives “on-demand” treatment for spontaneous bleeding episodes; wherein on demand” treatment may be factor replacement therapy. In one embodiment, prophylactic use of the pharmaceutical composition disclosed herein may reduce the ABR of a subject with a coagulopathy by 80%, preferably 90%, compared with on-demand treatment; wherein “on demand” treatment may be factor replacement therapy. In one embodiment, use of the pharmaceutical composition disclosed herein may result in the ABR of a subject with a coagulopathy being no greater than 5, such as no greater than 4, such as no greater than 3, such as no greater than 2, such as no greater than 1; preferably, the subject will have no spontaneous bleeds at all.

Preferably, prophylactic use of the pharmaceutical composition disclosed herein significantly reduces the ABR of a subject suffering from a coagulopathy selected from the group consisting of haemophilia A, haemophilia B, haemophilia A with inhibitors and/or haemophilia B with inhibitors. In one embodiment, use of the pharmaceutical composition disclosed herein may result in the ABR of such subject being no greater than 5, such as no greater than 4; such as no greater than 3, such as no greater than 2, such as no greater than 1; preferably, the subject will have no spontaneous bleeds at all. In one embodiment, prophylactic use of the pharmaceutical composition disclosed herein may reduce the ABR of a subject with haemophilia A by 80%, preferably 90%, compared with on-demand treatment; wherein on-demand treatment is factor replacement therapy. In one embodiment, prophylactic use of the pharmaceutical composition disclosed herein may reduce the ABR of a subject with haemophilia B by 80%, preferably 90%, compared with on-demand treatment; wherein on-demand treatment is factor replacement therapy. In one embodiment, prophylactic use of the pharmaceutical composition disclosed herein may reduce the ABR of a subject with haemophilia A with inhibitors by 80%, preferably 90%, compared with on-demand treatment. In one embodiment, prophylactic use of the pharmaceutical composition disclosed herein may reduce the ABR of a subject with haemophilia B with inhibitors by 80%, preferably 90%, compared with on-demand treatment.

On-demand, periodic or prophylactic use of the pharmaceutical composition disclosed herein may significantly reduce haemorrhage in women suffering from menorrhagia. Use of the pharmaceutical composition disclosed herein might even allow women of childbearing age suffering from menorrhagia to bear children, as such women would not have to rely on contraceptives to control their haemorrhage.

The pharmaceutical formulation disclosed herein may be administered via one or more routes of administration using one or more of a variety of methods known in the art. As will be appreciated by the skilled medical practitioner, the route and/or mode of administration will vary depending upon the desired results.

The pharmaceutical composition disclosed herein is suitable for parenteral administration. The phrase “parenteral administration” as used herein means modes of administration other than enteral and topical administration, usually by injection. The pharmaceutical formulation disclosed herein may be administered intravenously. The pharmaceutical formulation disclosed herein may be administered intramuscularly. Preferably, the pharmaceutical formulation disclosed herein is administered subcutaneously.

Suitable dosages of the pharmaceutical formulation disclosed herein may be determined by a skilled medical practitioner. The amount of the active pharmaceutical ingredient (API), concizumab, in the pharmaceutical composition disclosed herein may be varied to obtain the amount of active pharmaceutical ingredient which is effective to achieve the desired therapeutic response without being toxic to the subject. The selected dosage level will depend upon a variety of pharmacokinetic factors including the route of administration, the time of administration, the rate of excretion of the API (concizumab), the duration of the treatment, other drugs, compounds and/or materials used in combination with the pharmaceutical formulation disclosed herein, the age, sex, weight, condition, general health and prior medical history of the subject being treated, and similar factors well known in the medical arts.

When treatment with the pharmaceutical composition disclosed herein is initiated, the subject will typically receive one or more loading doses followed by a maintenance dosing scheme. Dosage regimens may be thereafter be adjusted to provide the optimum desired response (e.g., a therapeutic response). For example, a single bolus may be administered, several divided doses may be administered over time or the dose may be proportionally reduced or increased as indicated by the exigencies of the therapeutic situation.

The dosage and frequency of administration may vary depending on whether the treatment is prophylactic or therapeutic. The dosage and frequency of administration may vary depending on the duration of treatment that is desired. In prophylactic applications, a relatively low dosage may be administered at predefined intervals over a long period of time. In therapeutic applications, a relatively high dosage may be administered, for example, until the patient shows partial or complete amelioration of symptoms of disease.

The pharmaceutical formulation disclosed herein may be administered: approximately daily, approximately every other day, approximately every third day, approximately every fourth day, approximately every fifth day, approximately every sixth day; approximately every week, such as every 3, 4, 5, 6, 7, 8, 9 or 10 days. Preferably, the pharmaceutical formulation is administered once daily or once weekly. The pharmaceutical formulation disclosed herein may be administered approximately every eighth day; approximately every ninth day; approximately every tenth day; approximately every eleventh day; approximately every twelfth day; approximately every thirteenth day; approximately once a fortnight; such as every 8, 9, 10, 11, 12, 13 or 14 days.

A suitable dose of concizumab may be in the range of about 0.1-200 mg concizumab per kg body weight (mg/kg), such as 0.1-50 mg/kg, such as 50-100 mg/kg, such as 100-150 mg/kg, such as 150-200 mg/kg.

A suitable dose of concizumab may be in the range of 0.1-2 mg/kg, such as 0.1-0.3 mg/kg, such as 0.2-0.4 mg/kg, such as 0.4-0.6 mg/kg, such as 0.6-0.8 mg/kg, such as 0.8-1.0 mg/kg, such as 1.0-1.2 mg/kg, such as 1.2-1.4 mg/kg, such as 1.4-1.6 mg/kg, such as 1.6-1.8 mg/kg, such as 1.9-2.0 mg/kg. In one preferred embodiment, the dose of concizumab is 0.1-0.3 mg/kg, such as 0.15 mg/kg, such as 0.2 mg/kg, such as 0.25 mg/kg, such as 0.30 mg/kg. In a preferred embodiment, the dose of concizumab is 0.2-0.4 mg/kg, such as about 0.25 mg/kg, such as about 0.35 mg/kg. In another preferred embodiment, the dose of concizumab is 1.6-1.8 mg/kg, such as about 1.65 mg/kg, such as about 1.75 mg/kg.

A suitable dose of concizumab may be in the range of about 0.1-50 mg/kg, such as about 0.1-10 mg/kg, such as about 10-20 mg/kg, such as about 20-30 mg/kg, such as about 30-40 mg/kg, such as about 40-50 mg/kg. A suitable dose of concizumab may be in the range of about 2-3 mg/kg/day, such as about 4-5 mg/kg/day, such as about 5-6 mg/kg/day, such as about 6-7 mg/kg/day, such as about 7-8 mg/kg/day, such as about 8-9 mg/kg/day, such as about 9-10 mg/kg/day.

A suitable dose of concizumab may be in the range of about 50-100 mg/kg, such as about 60-90 mg/kg, such as about 50-60 mg/kg, 60-70 mg/kg, 70-80 mg/kg, 80-90 mg/kg, 90-100 mg/kg.

A suitable dose of concizumab may be in the range of about 100-150 mg/kg, such as about 110-140 mg/kg, such as about 100-110 mg/kg, such as about 110-120 mg/kg, such as about 120-130 mg/kg, such as about 130-140 mg/kg, such as about 140-150 mg/kg.

A suitable dose of concizumab may be in the range of about 150-200 mg/kg, such as about 160-190 mg/kg, such as about 150-160 mg/kg, such as about 160-170 mg/kg, such as about 170-180 mg/kg, such as about 180-190 mg/kg, such as about 190-200 mg/kg.

For prophylactic therapy, a suitable loading dose of concizumab may be in the range of about 0.1 μg/kg/day to about 200 mg/kg/day. In a preferred embodiment, a suitable loading dose of concizumab may be in the range of 0.75-2 mg/kg/day, such as about 1 mg/kg/day, such as about 1.75 mg/kg/day.

For prophylactic therapy, a suitable maintenance dose of concizumab may be in the range of from about 0.1 μg/kg/day to about 200 mg/kg/day. A suitable maintenance dosage may be from about 0.1 μg/kg/day to about 10 mg/kg/day. A suitable maintenance dose may be in the range of about 0.1 mg/kg/day to about 5 mg/kg/day. In a preferred embodiment, a suitable maintenance dose of concizumab may be in the range of 0.1-2 mg/kg/day, preferably 0.2-0.4 mg/kg/day, such as 0.25 mg/kg/day, such as 0.35 mg/kg/day. In another preferred embodiment, a suitable maintenance dose of concizumab may be in the range of 0.75-2 mg/kg/week, such as about 1.5-2.0 mg/kg/week, such as about 1.75 mg/kg/week.

For daily prophylactic therapy: a loading dose of 0.75-2 mg/kg, such as about 1 mg/kg may be administered on treatment day 1; a maintenance dose of 0.25 mg/kg may be administered daily from treatment day 2 and for at least 6 months, and a subject having had at least 2 spontaneous bleeds during the first 6 months of treatment may be dose escalated to 0.35 mg/kg/day. This may be a suitable dosage regime for subjects requiring constant treatment, such as subjects with a blood coagulation factor deficit, such as subjects with haemophilia A, haemophilia B, haemophilia A with inhibitors or haemophilia B with inhibitors.

For daily prophylactic therapy: a loading dose of 0.5-1 mg/kg, such as about 0.5 or about 1 mg/kg may be administered on treatment day 1; a maintenance dose of 0.2 mg/kg may be administered daily from treatment day 2 and for at least 4 weeks, optionally, with dose adjustment to 0.15 mg/kg, 0.2 mg/kg or 0.25 mg/kg at any point in time thereafter.

For daily prophylactic therapy: a loading dose of about 0.5-1 mg/kg, such as about 0.5 mg/kg or about 1 mg/kg, administered on treatment day 1; a maintenance dose of 0.1-0.25 mg/kg, such as 0.15-0.2 mg/kg, such as 0.15 mg/kg or 0.2 mg/kg, administered daily from treatment day 2; optionally, with dose adjustment to 0.15 mg/kg, 0.2 mg/kg or 0.25 mg/kg after the first 4 weeks of treatment.

For daily prophylactic therapy: a loading dose of 0.5 mg/kg administered on treatment day 1; followed by a dose of 0.15 mg/kg daily from treatment day 2, with dose escalation to 0.20 mg/kg if 3 or more spontaneous bleeding episodes occur within any 12 week period, after treatment day 2, and with further dose escalation to 0.25 mg/kg if 3 or more spontaneous bleeding episodes occur within any 12 week period, after treatment day 2.

For daily prophylactic therapy: a loading dose of about 0.5-1 mg/kg, such as about 0.5 mg/kg or about 1 mg/kg, administered on treatment day 1; a maintenance dose of 0.2 mg/kg administered daily from treatment day 2 and for at least 4 weeks; optionally, with dose adjustment to 0.15 mg/kg, 0.2 mg/kg or 0.25 mg/kg at any point in time thereafter.

In another embodiment, a loading dose of 0.75-2 mg/kg, such as about 1.75 mg/kg is administered on days 1 and 2 and a maintenance dose of 1.75 mg/kg/week is administered from day 8. This may be a suitable dosage regime for subjects requiring constant treatment, such as subjects with a blood coagulation factor deficit, such as subjects with haemophilia A, haemophilia B, haemophilia A with inhibitors or haemophilia B with inhibitors.

In a third embodiment, a single dose of 0.75-2 mg/kg, such as about 1.75 mg/kg, is administered one or two days before expected haemorrhage. This may be a suitable dosage regime for subjects requiring occasional or intermittent protection from excessive bleeding. For example, a woman with menorrhagia may benefit from such a single bolus injection of concizumab, preferably 1 or 2 days before menstruation starts.

In a fourth embodiment, a loading dose of 0.75-2 mg/kg, such as 1 mg/kg, is administered on day 1 and a maintenance dose of about 0.25 mg/kg is administered daily from day 2 and for up to 14 days, such as daily for 5, 6, 7, 8, 9, 10, 11, 12, 13 or 14 days. This may be a suitable dosage regime for subjects requiring occasional protection from excessive bleeding. For example, a woman with menorrhagia may benefit from such a dosing regimen during a menstrual cycle, wherein the loading dose would preferably be administered 1 or 2 days before menstruation starts.

There may be substantial individual variation in the concizumab plasma concentration of subjects that have received the same dose of concizumab and/or that benefit from the same therapeutic effect of the same dose. The amount of pharmaceutical composition administered to a subject may be such that the plasma concentration of concizumab in said subject is about 50 ng/ml to about 100 μg/ml, such as about 50 ng/ml to 1 μg/ml, such as about 1-5 μg/ml, such as about 5-10 μg/ml, such as about 10-15 μg/ml, such as about 15-20 μg/ml, such as about 20-25 μg/ml, such as about 25-30 μg/ml, such as about 30-35 μg/ml, such as about 35-40 μg/ml, such as about 40-45 μg/ml, such as about 45-50 μg/ml, such as about 50-55 μg/ml, such as about 55-60 μg/ml, such as about 60-65 μg/ml, such as about 65-70 μg/ml, such as 70-75 μg/ml, such as about 75-80 μg/ml, such as about 80-85 μg/ml, such as about 85-90 μg/ml, such as about 90-95 μg/ml, such as about 95-100 μg/ml. The relative distribution of the TFPI pools in different subjects may account for such individual variation.

Subjects may be treated with a pharmaceutical composition as described herein, wherein the composition comprises 5-150 mg/ml concizumab, such as about 150 mg/ml, such as about 140 mg/ml, such as about 130 mg/ml, such as about 120 mg/ml, such as about 110 mg/ml, such as 95-105 mg/ml, such as about 100 mg/ml, such as about 90 mg/ml, such as about 80 mg/ml concizumab, such as about 70 mg/ml concizumab, such as about 60 mg/ml concizumab, such as about 50 mg/ml concizumab, such as 45-55 mg/ml, such as about 40 mg/ml concizumab, such as about 30 mg/ml concizumab, such as about 20 mg/ml concizumab, such as 8-12 mg/ml, such as about 10 mg/ml concizumab, such as about 5 mg/ml. Subjects with higher body weights may find injections more comfortable if the pharmaceutical composition comprises concizumab in a higher concentration, such as 95-105 mg/ml, such as about 100 mg/kg, as the volume to be injected will be smaller. In contrast, the dosing of infants may be more accurate if the pharmaceutical composition comprises a lower concentration of concizumab, such as 10 mg/ml.

The pharmaceutical formulation disclosed herein may be co-administered with one or more other therapeutic agents. The other agent may be an agent that complements or enhances the pro-coagulant effect of the pharmaceutical formulation disclosed herein. The other agent may be an agent that acts to enhance blood coagulation, such as a blood coagulation factor; such as a recombinantly produced blood coagulation factor.

The other agent may be temporarily administered when subjects are being switched from their current therapy to that with the pharmaceutical formulation disclosed herein. For example, subjects with haemophilia A that are being switched from Factor VIII prophylactic therapy to therapy with the pharmaceutical formulation disclosed herein may receive both treatments for 2 weeks.

In subjects receiving prophylactic treatment with the pharmaceutical formulation disclosed herein, the other therapeutic agent may be for the treatment of so-called “breakthrough bleeds”. In subjects with haemophilia A or B with inhibitors, breakthrough bleeds may be treated with, for example, activated prothrombin complex concentrates (aPCC) such as FEIBA®; a combination of activated Factor VII (FVIIa) and Factor X (FX) such as Byclot®; or a Factor Vila product such as NovoSeven®. Hence, the pharmaceutical formulation disclosed herein may be co-administered with Factor Vila. In subjects with haemophilia A, breakthrough bleeds may be treated with Factor VIII. Hence, the pharmaceutical formulation disclosed herein may be co-administered with Factor VIII. In subjects with haemophilia B, breakthrough bleeds may be treated with Factor IX. Hence, the pharmaceutical formulation disclosed herein may be co-administered with Factor IX. Subjects with a coagulopathy may benefit from treatment with the pharmaceutical formulation disclosed herein and an antifibrinolytic drug, such as aminocaproic acid or tranexamic acid. In one embodiment, subjects with haemophilia undergoing tooth extraction or surgery may benefit from such a combination therapy. In another embodiment, subjects with menorrhagia may benefit from such a combination therapy.

Combined administration of two or more agents may be achieved in several different ways. In one embodiment, the pharmaceutical formulation disclosed herein and the other therapeutic agent may be administered in separate compositions, as part of a combined therapy. The pharmaceutical formulation disclosed herein may be administered before, after or concurrently with the other therapeutic agent, such that they are simultaneously present in vivo.

Following is a non-limiting list of embodiments of the present invention.

### EMBODIMENTS

1. A method of preparing a pharmaceutical formulation of concizumab, comprising the following steps:
   obtaining or preparing a drug substance (DS) comprising concizumab as the active pharmaceutical ingredient and having a pH of 5.0-6.5, such as pH 5.5-6.5, preferably about pH 6.0;
   preparing an aqueous, surfactant-free excipient solution comprising one or two antimicrobial preservatives, optionally selected from the group consisting of phenol, m-cresol, benzyl alcohol and chlorobutanol, and having a pH of 5.0-6.5, such as pH 5.5-6.5, preferably about 6.0;
   optionally, adjusting the pH of the excipient solution;
   mixing the DS and the excipient solution;
   adding surfactant to the mixture of the DS and excipient solution;
   optionally, adding water.
2. A method of preparing a pharmaceutical formulation of concizumab, comprising:
   obtaining or preparing a drug substance (DS) comprising concizumab as the active pharmaceutical ingredient (API) and having a pH of 5.0-6.5, such as pH 5.5-6.5, preferably about pH 6.0;
   preparing an aqueous, surfactant-free excipient solution comprising benzyl alcohol and having a pH of 5.0-6.5, such as pH 5.5-6.5, preferably about pH 6.0;
   optionally, adjusting the pH of the excipient solution;
   mixing the DS and the excipient solution, such that the concentration of benzyl alcohol in the DS-excipient mixture is less than 35 mg/ml, such as 20 mg/ml or less;
   adding Polysorbate 20 or Polysorbate 80 to the mixture of the DS and excipient solution;
   optionally, adding water.
3. A method of preparing a pharmaceutical formulation of concizumab, comprising:
   obtaining or preparing a drug substance (DS) comprising concizumab as the active pharmaceutical ingredient (API) and having a pH of 5.0-6.5, such as pH 5.5-6.5, preferably about pH 6.0;
   preparing an aqueous, surfactant-free excipient solution comprising benzyl alcohol and having a pH of 5.0-6.5, such as pH 5.5-6.5, preferably about pH 6.0;
   optionally, adjusting the pH of the excipient solution;
   mixing the DS and the excipient solution, such that the concentration of benzyl alcohol in the DS-excipient mixture is 35 mg/ml or less, such as 20 mg/ml or less;
   adding Poloxamer 188 to the mixture of the DS and excipient solution;
   optionally, adding water.
4. A method of preparing a pharmaceutical formulation of concizumab, comprising:
   obtaining or preparing a drug substance (DS) comprising concizumab as the active pharmaceutical ingredient (API) and having a pH of 5.0-6.5, such as pH 5.5-6.5, preferably about pH 6.0;
   preparing an aqueous, surfactant-free excipient solution comprising chlorobutanol and having a pH of 5.0-6.5, such as pH 5.5-6.5, preferably about pH 6.0;
   optionally, adjusting the pH of the excipient solution;
   mixing the DS and the excipient solution, such that the concentration of chlorobutanol in the DS-excipient mixture is 7.5 mg/ml or less, such as 6.5 mg/ml or less;
   adding Polysorbate 20, Polysorbate 80 or Poloxamer 188 to the mixture of the DS and excipient solution;
   optionally, adding water.
5. A method of preparing a pharmaceutical formulation of concizumab, comprising:
   obtaining or preparing a drug substance (DS) comprising concizumab as the active pharmaceutical ingredient (API) and having a pH of 5.0-6.5, such as pH 5.5-6.5, preferably about pH 6.0;
   preparing an aqueous, surfactant-free excipient solution comprising m-cresol and having a pH of 5.0-6.5, such as pH 5.5-6.5, preferably about pH 6.0;
   optionally, adjusting the pH of the excipient solution;
   mixing the DS and the excipient solution, such that the concentration of m-cresol in the DS-excipient mixture is less than 3 mg/ml;
   adding Polysorbate 20 to the mixture of the DS and excipient solution;
   optionally, adding water.
6. A method of preparing a pharmaceutical formulation of concizumab, comprising:
   obtaining or preparing a drug substance (DS) comprising concizumab as the active pharmaceutical ingredient (API) and having a pH of 5.0-6.5, such as pH 5.5-6.5, preferably about pH 6.0;
   preparing an aqueous, surfactant-free excipient solution comprising m-cresol and having a pH of 5.0-6.5, such as pH 5.5-6.5, preferably about pH 6.0;
   optionally, adjusting the pH of the excipient solution;
   mixing the DS and the excipient solution, such that the concentration of m-cresol in the DS-excipient mixture is no greater than 5 mg/ml;
   adding Polysorbate 80 to the mixture of the DS and excipient solution;
   optionally, adding water.
7. A method of preparing a pharmaceutical formulation of concizumab, comprising:
   obtaining or preparing a drug substance (DS) comprising concizumab as the active pharmaceutical ingredient (API) and having a pH of 5.0-6.5, such as pH 5.5-6.5, preferably about pH 6.0;
   preparing an aqueous, surfactant-free excipient solution comprising m-cresol and having a pH of 5.0-6.5, such as pH 5.5-6.5, preferably about pH 6.0;
   optionally, adjusting the pH of the excipient solution;
   mixing the DS and the excipient solution, such that the concentration of m-cresol in the DS-excipient mixture is less than 10 mg/ml;
   adding Poloxamer 188 to the mixture of the DS and excipient solution;
   optionally, adding water.
8. A method of preparing a pharmaceutical formulation of concizumab, comprising:
   obtaining or preparing a drug substance (DS) comprising concizumab as the active pharmaceutical ingredient (API) and having a pH of 5.0-6.5, such as pH 5.5-6.5, preferably about pH 6.0;
   preparing an aqueous, surfactant-free excipient solution comprising phenol and having a pH of 5.0-6.5, such as pH 5.5-6.5, preferably about pH 6.0;
   optionally, adjusting the pH of the excipient solution;
   mixing the DS and the excipient solution, such that the concentration of phenol in the DS-excipient mixture is less than 6 mg/ml;
   adding Polysorbate 20 to the mixture of the DS and excipient solution;
   optionally, adding water.
9. A method of preparing a pharmaceutical formulation of concizumab, comprising:
   obtaining or preparing a drug substance (DS) comprising concizumab as the active pharmaceutical ingredient (API) and having a pH of 5.0-6.5, such as pH 5.5-6.5, preferably about pH 6.0;
   preparing an aqueous, surfactant-free excipient solution comprising less than 55 mg/ml, such as no more than 45 mg/ml phenol, such as about 4-45 mg/ml phenol, and having a pH of 5.0-6.5, such as pH 5.5-6.5, preferably about pH 6.0;
   optionally, adjusting the pH of the excipient solution;
   mixing the DS and the excipient solution, such that the concentration of phenol in the DS-excipient mixture is less than 8 mg/ml;
   adding Polysorbate 80 to the mixture of the DS and excipient solution;
   optionally, adding water.
10. A method of preparing a pharmaceutical formulation of concizumab, comprising:
   obtaining or preparing a drug substance (DS) comprising concizumab as the active pharmaceutical ingredient (API) and having a pH of 6.0;
   preparing an aqueous, surfactant-free excipient solution comprising L-arginine HCl, L-histidine, sucrose, sodium chloride, phenol and water, such that the concentration of phenol is less than 55 mg/ml, such as no greater than 45 mg/ml, and adjusting the pH to 6.0;
   mixing the DS and the excipient solution, such that the concentration of phenol in the mixture is less than 8 mg/ml, such as about 4.0-7.5 mg/ml, such as about 4.0-5.5 mg/ml, such as about 5.5-7.5, such as about 5.5-6.5, preferably about 6 mg/ml;
   adding polysorbate 80 to the mixture to a final concentration of 0.1-2.0 mg/ml, such as 0.1-0.3 mg/ml, preferably about 0.25 mg/ml;
   optionally, adding WFI to the final batch volume.
11. A method of preparing a pharmaceutical formulation of concizumab, comprising:
   obtaining or preparing a drug substance (DS) comprising concizumab as the active pharmaceutical ingredient (API) and having a pH of 5.0-6.5, such as pH 5.5-6.5, preferably about pH 6.0;
   preparing an aqueous, surfactant-free excipient solution comprising phenol and having a pH of 5.0-6.5, such as pH 5.5-6.5, preferably about pH 6.0;
   optionally, adjusting the pH of the excipient solution;
   mixing the DS and the excipient solution, such that the concentration of phenol in the DS-excipient mixture is less than 10 mg/ml;
   adding Poloxamer 188 to the mixture of the DS and excipient solution;
   optionally, adding water.
12. A method of preparing a pharmaceutical formulation of concizumab, comprising:
   obtaining or preparing a drug substance (DS) comprising concizumab as the active pharmaceutical ingredient (API) and having a pH of 6.0;
   preparing an excipient solution comprising L-arginine HCl, L-histidine, sucrose, sodium chloride, phenol and water, such that the concentration of phenol in the solution is less than 55 mg/ml, such as no greater than about 45 mg/ml, such as about such as about 6-45 mg/ml, and adjusting the pH to 6.0;
   optionally, adjusting the pH of the excipient solution;
   adding DS to the excipient solution, such that the concentration of phenol in the mixture is no greater than about 6 mg/ml;
   adding polysorbate 80 to the mixture, to a final concentration of 0.1-0.3 mg/ml;
   optionally, adding water.
13. The method according to any one of the preceding embodiments, wherein the drug substance comprising concizumab is a liquid composition.
14. The method according to any one of the preceding embodiments, wherein the drug substance comprises 80-300 mg/ml concizumab, preferably 100-140 mg/ml concizumab.
15. The method according to any one of the preceding embodiments, wherein the drug substance comprising concizumab further comprises a buffer, a tonicity agent, a viscosity lowering agent and a stabiliser.
16. The method according to any one of the preceding embodiments, wherein the drug substance comprising concizumab further comprises a buffer, a tonicity agent and at least one salt.
17. The method according to any one of the preceding embodiments, wherein the drug substance comprising concizumab further comprises a buffer, a tonicity agent, an organic salt and an inorganic salt.
18. The method according to any one of the preceding embodiments, wherein the drug substance comprising concizumab comprises histidine as a buffer, sucrose as a tonicity agent, sodium chloride as a viscosity lowering agent and arginine or arginine hydrochloride as a stabiliser.
19. The method according to any one of the preceding embodiments, wherein the drug substance and the aqueous, surfactant-free excipient solution comprise the same buffer(s), tonicity agent(s), viscosity lowering agent and stabiliser.
20. The method according to any one of the preceding embodiments, wherein the drug substance and the aqueous, surfactant-free excipient solution comprise the same buffer(s), tonicity agent(s) and salt(s).
21. The method according to any one of the preceding embodiments, wherein the aqueous, surfactant-free excipient solution comprises one or two antimicrobial preservatives, histidine, sucrose, arginine hydrochloride and sodium chloride.
22. The method according to any one of the preceding embodiments, wherein the antimicrobial preservative(s) is/are selected from the group consisting of benzyl alcohol, chlorobutanol, m-cresol and phenol.
23. The method according to any one of the preceding embodiments, wherein the aqueous, surfactant-free excipient solution comprises one antimicrobial preservative.
24. The method according to any one of embodiments 1-3, 13-23, wherein the antimicrobial preservative is benzyl alcohol.
25. The method according to any one of embodiments 1-3, 13-23, wherein the antimicrobial preservative is benzyl alcohol and wherein the concentration of the benzyl alcohol in the mixture of the DS and the excipient solution does not exceed 20 mg/ml.
26. The method according to any one of embodiments 1-3, 13-23, wherein the pharmaceutical formulation of concizumab contains about 9-11 mg/ml benzyl alcohol, such as about 10 mg/ml benzyl alcohol.
27. The method according to any one of embodiments 1, 4, 13-23, wherein the antimicrobial preservative is chlorobutanol.
28. The method according to any one of embodiments 1, 4, 13-23, 27, wherein the preservative is chlorobutanol and wherein the concentration of the chlorobutanol in the mixture of the DS and the excipient solution does not exceed 7.5 mg/ml.
29. The method according to any one of embodiments 1, 4, 13-23, 27-28, wherein the pharmaceutical formulation of concizumab contains about 3.0 to 7.5 mg/ml chlorobutanol, such as about 5 mg/ml chlorobutanol.
30. The method according to any one of embodiments 1, 5-7, 13-23, wherein the antimicrobial preservative is m-cresol.
31. The method according to any one of embodiments 1, 5-7, 13-23, 30, wherein the preservative is m-cresol and wherein the concentration of the antimicrobial preservative in the mixture of the DS and the excipient solution is less than 10 mg/ml, such as less than 5 mg/ml, such as about 3 mg/ml or less.
32. The method according to any one of embodiments 1, 5-7, 13-23, 30-31, wherein the pharmaceutical formulation of concizumab contains about 2.7-3.2 mg/ml m-cresol, such as about 3 mg/ml m-cresol.
33. The method according to any one of embodiments 1, 8-23, wherein the antimicrobial preservative is phenol.
34. The method according to any one of embodiments 1, 8-23, 33, wherein the preservative is phenol and wherein the concentration of phenol in the aqueous, surfactant-free excipient solution is less than 55 mg/ml, such as no more than 45 mg/ml, such as about 4-45 mg/ml, such as about 4-40 mg/ml, such as about 4-10 mg/ml, such as about 11-20 mg/ml, such as about 21-30 mg/ml, such as about 31-40 mg/ml, such as about 41-45 mg/ml; such as about 11-15 mg/ml, such as about 16-20 mg/ml, such as about 21-25 mg/ml, such as about 26-30 mg/ml, such as about 31-35 mg/ml, such as about 36-40 mg/ml, such as about 41-45 mg/ml.
35. The method according to any one of embodiments 1, 8-23, 33-34, wherein the preservative is phenol, wherein the concentration of phenol in the mixture of the DS and the excipient solution is at least 2.5 mg/ml and less than about 10 mg/ml, such as less than about 8 mg/ml, such as no greater than about 6 mg/ml; such as less than about 6 mg/ml; such as about 2.5-5.0 mg/ml; such as about 3.0-5.0 mg/ml; such as about 3.0-4.0 mg/ml; such as about 3.0 mg/ml, such as least about 3.2 mg/ml, such as about 3.5 mg/ml, such as about 4.0-7.5 mg/ml, such as about 4.0-5.5 mg/ml, such as about 4.0-5.0 mg/ml, such as about 4.5-5.5 mg/ml, such as about 5.5-7.5, such as about 5.5-6.5, such as about 6 mg/ml; such as less than about 6 mg/ml; and wherein the concentration of concizumab in the pharmaceutical formulation is 10 mg/ml.
36. The method according to any one of embodiments 1, 8-23, 33-35, wherein the preservative is phenol, wherein the concentration of phenol in the mixture of the DS and the excipient solution is at least 2.5 mg/ml and less than about 10 mg/ml, such as less than about 8 mg/ml, such as no greater than about 6 mg/ml; such as less than about 6 mg/ml; such as about 2.5-5.0 mg/ml; such as about 3.0-5.0 mg/ml; such as about 3.0-4.0 mg/ml; such as about 3.0 mg/ml, such as least about 3.2 mg/ml, such as about 3.5 mg/ml, such as about 4.0-7.5 mg/ml, such as about 4.0-5.5 mg/ml, such as about 4.0-5.0 mg/ml, such as about 4.5-5.5 mg/ml, such as about 5.5-7.5, such as about 5.5-6.5, such as about 6 mg/ml; and wherein the concentration of concizumab in the pharmaceutical formulation is about 40 mg/ml.
37. The method according to any one of embodiments 1, 8-23, 33-36, wherein the preservative is phenol, wherein the concentration of phenol in the mixture of the DS and the excipient solution is at least 2.5 mg/ml and less than about 10 mg/ml, such as less than about 8 mg/ml, such as no greater than about 6 mg/ml; such as less than about 6 mg/ml; such as about 2.5-5.0 mg/ml; such as about 3.0-5.0 mg/ml; such as about 3.0-4.0 mg/ml; such as about 3.0 mg/ml, such as least about 3.2 mg/ml, such as about 3.5 mg/ml, such as about 4.0-7.5 mg/ml, such as about 4.0-5.5 mg/ml, such as about 4.0-5.0 mg/ml, such as about 4.5-5.5 mg/ml, such as about 5.5-7.5, such as about 5.5-6.5, such as about 6 mg/ml; and wherein the concentration of concizumab in the pharmaceutical formulation is about 100 mg/ml.
38. The method according to any one of embodiments 1, 8-23, 33-37, wherein the concentration of phenol, in the mixture of the DS and the excipient solution, does not exceed 10 mg/ml, such as 6 mg/ml.
39. The method according to any one of embodiments 1, 8-23, 33-38, wherein the concentration of phenol in the pharmaceutical formulation is 3-6 mg/ml, preferably 3-4 mg/ml, even more preferably about 3.5 mg/ml.
40. The method according to any one of the preceding embodiments, wherein the surfactant is selected from the group consisting of polysorbate 20, polysorbate 80 and poloxamer 188.
41. The method according to any one of the preceding embodiments, wherein the surfactant is polysorbate 20.
42. The method according to any one of the preceding embodiments, wherein the pharmaceutical formulation comprises 0.1-2.0 mg/ml, such as 0.1-0.3 mg/ml polysorbate 20, such as 0.1-0.2 mg/ml, such as 0.2-0.3 mg/ml, such as about 0.25 mg/ml polysorbate 20.
43. The method according to any one of the preceding embodiments, wherein the pharmaceutical formulation comprises about 0.25 mg/ml polysorbate 20.
44. The method according to any one of embodiments 1-40, wherein the surfactant is polysorbate 80.
45. The method according to any one of embodiments 1-40, 44, wherein the pharmaceutical formulation comprises 0.1-2.0 mg/ml polysorbate 80, such as 0.1-0.3 mg/ml, such as 0.1-0.2 mg/ml, such as 0.2-0.3 mg/ml, such as about 0.25 mg/ml polysorbate 80.
46. The method according to any one of embodiments 1-40, 44-45, wherein the pharmaceutical formulation comprises about 0.25 mg/ml polysorbate 80.
47. The method according to any one of embodiments 1-40, wherein the surfactant is poloxamer 188.
48. The method according to any one of embodiments 1-40, 47, wherein the pharmaceutical formulation comprises 0.1-10.0 mg/ml poloxamer 188, such as 0.9-1.1, such as about 1.0 mg/ml poloxamer 188.
49. The method according to any one of the preceding embodiments, wherein the drug substance, the excipient solution, the mixture of the drug substance and excipient solution and the pharmaceutical formulation comprise a tonicity modifying agent which is sucrose, mannitol or propylene glycol.
50. The method according to any one of the preceding embodiments, wherein the drug substance, the excipient solution, the mixture of the drug substance and excipient solution and the pharmaceutical formulation comprise a tonicity modifying agent which is sucrose.
51. The method according to any one of the preceding embodiments, wherein the pharmaceutical formulation of concizumab comprises between 50 and 250 mM of a tonicity modifying agent, such as about 150 mM sucrose.
52. The pharmaceutical formulation obtained by the method according to any one of the preceding embodiments.
53. The pharmaceutical formulation obtained by the method according to any one of the preceding embodiments and comprising 5-110 mg/ml, such as 5-15 mg/ml, such as about 10 mg/ml, such as 15-25 mg/ml, such as about 20 mg/ml, such as 25-35 mg/ml, such as about 30 mg/ml, such as 35-45 mg/ml, such as about 40 mg/ml, such as 45-55 mg/ml, such as about 50 mg/ml, such as 55-65 mg/ml, such as about 60 mg/ml, such as 65-75 mg/ml, such as about 70 mg/ml, such as 75-85 mg/ml, such as about 80 mg/ml, such as 85-95 mg/ml, such as about 90 mg/ml, such as 95-105 mg/ml, such as about 100 mg/ml concizumab.
54. The pharmaceutical formulation of concizumab according to any one of the preceding embodiments, which is isotonic.
55. The pharmaceutical formulation according to any one of the preceding embodiments, comprising concizumab, histidine, sucrose, sodium chloride, arginine hydrochloride, polysorbate 80 and having a pH of 5.5-6.5, preferably about 6.0.
56. The pharmaceutical formulation according to any one of embodiments 52-55, comprising:
   Concizumab: 5-110 mg/ml, such as about 10, 40 or 100 mg/ml
   Phenol: about 0.35% (3.5 mg/ml)
   L-Histidine: about 33 mM (5.12 mg/ml)
   Sodium chloride: about 25 mM (1.46 mg/ml)
   L-Arginine HCl: about 25 mM (5.27 mg/ml)
   Sucrose: about 150 mM (51.3 mg/ml)
   Polysorbate 80: about 0.25 mg/ml
   water
   pH about 6.0
57. The pharmaceutical formulation according to any one of embodiments 52-56, comprising:
   Concizumab: about 100 mg/ml
   Phenol: about 0.35% (3.5 mg/ml)
   L-Histidine: about 33 mM (5.12 mg/ml)
   Sodium chloride: about 25 mM (1.46 mg/ml)
   L-Arginine HCl: about 25 mM (5.27 mg/ml)
   Sucrose: about 150 mM (51.3 mg/ml)
   Polysorbate 80: about 0.25 mg/ml
   water
   pH about 6.0
58. The pharmaceutical formulation according to any one of embodiments 52-56, comprising:
   Concizumab: about 40 mg/ml
   Phenol: about 0.35% (3.5 mg/ml)
   L-Histidine: about 33 mM (5.12 mg/ml)
   Sodium chloride: about 25 mM (1.46 mg/ml)
   L-Arginine HCl: about 25 mM (5.27 mg/ml)
   Sucrose: about 150 mM (51.3 mg/ml)
   Polysorbate 80: about 0.25 mg/ml
   water
   pH about 6.0
59. The pharmaceutical formulation according to any one of embodiments 52-56, comprising:
   Concizumab: about 10 mg/ml
   Phenol: about 0.35% (3.5 mg/ml)
   L-Histidine: about 33 mM (5.12 mg/ml)
   Sodium chloride: about 25 mM (1.46 mg/ml)
   L-Arginine HCl: about 25 mM (5.27 mg/ml)
   Sucrose: about 150 mM (51.3 mg/ml)
   Polysorbate 80: about 0.25 mg/ml
   water
   pH about 6.0
60. The pharmaceutical formulation according to any one of embodiments 52-59 for use in the treatment of a coagulopathy.
61. The pharmaceutical formulation according to any one of embodiments 52-59 for use in the treatment of an acquired and/or congenital and/or iatrogenic coagulopathy.
62. The pharmaceutical formulation according to any one of embodiments 52-59 for use in the treatment of an acquired coagulopathy.
63. The pharmaceutical formulation according to any one of embodiments 52-59 for use in the treatment of a congenital coagulopathy.
64. The pharmaceutical formulation according to any one of embodiments 52-59 for use in the treatment of a coagulopathy that is partly congenital and partly acquired.
65. The pharmaceutical formulation according to any one of embodiments 52-59 for use in the treatment of an iatrogenic coagulopathy
66. The pharmaceutical formulation according to any one of embodiments 52-59 for use in the treatment of a disease that may ensue from a congenital and/or acquired and/or iatrogenic coagulopathy.
67. The pharmaceutical formulation according to any one of embodiments 52-59 for use in the treatment of haemorrhage.
68. The pharmaceutical formulation according to any one of embodiments 52-59 for use in the treatment of a coagulopathy selected from the group consisting of haemophilia A, haemophilia A with inhibitors (allo-antibodies against Factor VIII), haemophilia B, haemophilia B with inhibitors (allo-antibodies against Factor IX), von Willebrand's disease, Glansmann's thrombasthenia and Bernard-Soulier syndrome.
69. The pharmaceutical formulation according to any one of embodiments 52-59 for use in the treatment of haemophilia A, haemophilia A with inhibitors (allo-antibodies against Factor VIII), haemophilia B and/or haemophilia B with inhibitors (allo-antibodies against Factor IX).
70. The pharmaceutical formulation for use according to any one of embodiments 60-69 wherein the pharmaceutical formulation is subcutaneously administered.
71. A prophylactically effective amount of the pharmaceutical formulation for use according to any one of embodiments 60-69.
72. A therapeutically effective amount of the pharmaceutical formulation for use according to any one of embodiments 60-69.
73. The pharmaceutical formulation for use according to any one of embodiments 60-69, wherein said pharmaceutical formulation is administered approximately once daily.
74. The pharmaceutical formulation for use according to any one of embodiments 60-69, wherein said pharmaceutical formulation is administered approximately once weekly.
75. The pharmaceutical formulation for use according to any one of embodiments 60-69, wherein said pharmaceutical formulation is administered once a fortnight.
76. The pharmaceutical formulation according to any one of embodiments 52-59, and one or more other therapeutic agents, such as Factor Vila, for use according to any one of embodiments 60-69.
77. The pharmaceutical formulation for use according to any one of embodiments 60-76, wherein the dose administered is in the range of 0.1-200 mg/kg, such as 0.1-50 mg/kg, such as 50-100 mg/kg, such as 100-150 mg/kg, such as 150-200 mg/kg.
78. The pharmaceutical formulation for use according to any one of embodiments 60-76, wherein:
   a loading dose of 0.75-2 mg concizumab per kg body weight, such as about 1 mg concizumab per kg body weight, may be administered on day 1;
   a maintenance dose of 0.25 mg concizumab per kg body weight may be administered daily from day 2 and for at least 6 months.
79. The pharmaceutical formulation for use according to embodiment 78, further comprising dose escalation to 0.35 mg concizumab per kg body weight per day after the first 6 months.
80. The pharmaceutical formulation for use according to any one of embodiments 60-77, wherein
   a loading dose of 0.75-2 mg concizumab per kg body weight, such as about 1.75 mg concizumab per kg body weight, is administered on days 1 and 2 and
   a maintenance dose of 1.75 mg concizumab per kg body weight per week is administered from day 8.
81. The pharmaceutical formulation for use according to any one of embodiments 60-77, wherein a single dose of 0.75-2 mg concizumab per kg body weight, such as about 1.75 mg concizumab per kg body weight, is administered one or two days before expected haemorrhage.
82. The pharmaceutical formulation for use according to any one of embodiments 60-77, wherein:
   a loading dose of 0.75-2 mg concizumab per kg body weight, such as 1 mg concizumab per kg body weight, is administered on day 1; and
   a maintenance dose of about 0.25 mg concizumab per kg body weight is administered daily from day 2 and for up to 14 days, such as daily for 5, 6, 7, 8, 9, 10, 11, 12, 13 or 14 days.
83. The pharmaceutical formulation for use according to any one of embodiments 60-77, wherein:
   a loading dose of about 0.5-1 mg/kg, such as about 0.5 mg/kg or about 1 mg/kg, is administered on treatment day 1; and
   a maintenance dose of 0.2 mg/kg is administered daily from treatment day 2 and for at least 4 weeks;
   optionally, with dose adjustment to 0.15 mg/kg, 0.2 mg/kg or 0.25 mg/kg daily, at any point in time thereafter

The present invention is further illustrated by the following examples which should not be construed as further limiting. The contents of all figures and all references, patents and published patent applications cited throughout this application are expressly incorporated herein by reference.

### EXAMPLES

### Analytical Methods

Various analytical methods, well known to the person skilled in the art, are used to assess the amounts of aggregates and other impurities in compositions such as the aqueous, surfactant-free excipient solution and the pharmaceutical formulation comprising concizumab. Appropriate analytical methods are provided in Assays I to IV, below.

### Assay I: Appearance

The appearance of the liquid formulation is determined by visual inspection of a sample, a method which is in accordance with Ph. Eur. The samples were compared to the clarity of a set of reference suspensions, each having a predetermined NTU (nephelometric turbidity units) value, which increases with increased turbidity.

### Assay II: High Molecular Weight Protein (HMWP) by SE-HPLC

SE-HPLC is used to determine HMWP in the drug product. It is performed in the same analytical run as the determination of the content of the API, in this case concizumab. HMWP is defined as the sum of the early eluting peaks, consisting of polymer and dimer forms of concizumab, with the dimer form consistently being the dominant form and the polymer being constant near the limit of quantification (LOQ), enabling a limit on the sum. It is measured as percentage HMWP of the total area.

### Assay III: ClarioStar Plate Reader

A ClarioSTAR plate reader is used to determine changes in the opalescence of solutions. The results are given as changes in light intensity, which can be used to determine interactions between the components in the solutions.

### Assay IV: Quantification of Subvisible Particles by Microflow Imaging (MFI)

MFI is used to determine the size and number of subvisible particles in the drug product. In MFI, bright-field images are captured in successive frames as a continuous sample stream passes through a flow-cell positioned in the field of view of a microscopic system. The digital images of the particles present in the sample are processed by image morphology analysis software that allows their quantification in size and count. Results are given as number of particles in sizes <2 μm, <5 μm, <10 μm and <25 μm.

### Example 1: Concizumab Interacts with Preservatives

The active pharmaceutical ingredient, concizumab, can interact with preservatives and form unwanted impurities in the formulation comprising it.

In this experiment, the method disclosed in the current application (embodied by manufacturing method 1 in example 6) was employed. The purpose of the experiment was to establish the concentration of preservative, in the excipient solution to be added to the drug substance, which would result in the preservative interacting with the antibody. Six excipient solutions, containing different concentrations of preservative, were tested as specified in table 1.

The tested batches of drug products contained concizumab (100 mg/ml), L-Arginine-HCl (5.27 mg/ml), L-Histidine (5.12 mg/ml), Sodium Chloride (1.46 mg/ml), Sucrose (51.3 mg/ml), Polysorbate 80 (0.25 mg/ml) and Phenol (2.5-7.5 mg/ml), and had a pH of 6.0 in aqueous solution. Tested batches were prepared according to the claimed manufacturing method (manufacturing method 1 in example 6)

A drug substance (DS) comprising concizumab and having a pH of 6.0, was prepared using the downstream process described in WO2009/138484. The concentration of concizumab in this drug substance was 139 mg/ml. Surfactant-free excipient solutions were prepared by dissolving L-Arginine HCl, L-Histidine, Sucrose, Sodium Chloride and Phenol in Water For Injection (10% of the final concizumab drug product volume). These solutions contained 25-75 mg/ml Phenol. The pH was adjusted to 6.0 using 2N Hydrochloric acid and/or 2N Sodium Hydroxide and the solution was homogenized. The drug substance was added whilst mixing and, finally, Polysorbate 80 was added to the mixture of the excipient solution and DS. This solution was 75% of the final concizumab drug product volume and contained 3.3-10 mg/ml Phenol and 0.33 mg/ml Polysorbate 80. Water was added to 100% of the final concizumab drug product volume (to achieve the pre-defined concentration of concizumab and all excipients in the drug product). The formulations were mixed and homogenized before being sterile filtered and filled in 1.5 ml cartridges.

The applied drug substance batch and the drug product batches after manufacture were assessed by analysing for HMWP, as described in Assay II. The results and the calculated increase in HMWP during the formulation is presented in table 1.

TABLE 1
Results for HMWP

| | |
|---|---|
| Phenol | HMWP % |

| | concentration | | | Increase |
|---|---|---|---|---|
| | in the excipient | Drug | Drug | during |
| Batch | solution | Substance | Product | manufacture |
| 1 | 25 mg/ml | 0.7 | 0.9 | 0.2 |
| 2 | 35 mg/ml | 0.7 | 0.9 | 0.2 |
| 3 | 45 mg/ml | 0.7 | 0.9 | 0.2 |
| 4 | 55 mg/ml | 0.7 | 1.1 | 0.4 |
| 5 | 65 mg/ml | 0.7 | 1.4 | 0.7 |
| 6 | 75 mg/ml | 0.7 | 2.7 | 2.0 |

For batches 5 and 6, heavy, insoluble precipitation was observed upon addition of drug substance, which was removed by filtration before measurement of HMWP.

The results for batches 1-3 show a similar increase in % HMWP during the formulation process, while batches 4-6 show an increase in the formation of HMWP with increasing phenol concentration in the excipient solution and final formulation. The results show that a phenol concentration of 55 mg/ml or more, in the excipient solution, leads to heavy, insoluble precipitation during preparation and an increase in the formation of HMWP during the formulation process.

From the results in this example it is evident that the concentration of phenol in the solution used to mix with preformulated drug substance should be below 55 mg/ml, or should not exceed about 45 mg/ml. Using a higher concentration will negatively affect the quality of the final pharmaceutical formulation.

### Example 2: Preservatives and Surfactants Interact to Form Unwanted Opalescence

Preservatives and surfactants can interact to form unwanted opalescence in a formulation. This is also demonstrated in example 3.

In this example, potential interactions between surfactants and preservatives were investigated. The tested compositions each contained one preservative and one surfactant. Commonly used surfactants, Polysorbate 20 (PS20), Polysorbate 80 (PS80) and Poloxamer 188 (PLX188), were tested together with commonly used preservatives, phenol, m-cresol, chlorobutanol and benzyl alcohol.

One concentration of each surfactant was selected as being representative of those commonly used (see table 2), while a range of commonly used concentrations of each preservative was selected (see table 3). (V. Gervasi et al, European Journal of Pharmaceutics and Biopharmaceutics 131 (2018)) 8-24 discusses commonly used concentrations.

TABLE 2
Selected surfactants and concentrations

| | Commonly used | |
|---|---|---|
| Surfactant | concentrations | Tested concentration |
| Polysorbate 20 | 0.01 mg/ml-0.5 mg/ml | 0.25 mg/ml |
| Polysorbate 80 | | 0.25 mg/ml |
| Poloxamer 188 | 0.5 mg/ml to 3 mg/ml | 1.0 mg/ml |

TABLE 3
Selected preservatives, common concentrations and tested range

| | Commonly used | |
|---|---|---|
| | concentration in drug | Tested concentration |
| Preservative | product | range |
| Phenol | 2.5 mg/ml-5.0 mg/ml | 4 mg/ml-16 mg/ml |
| m-cresol | 2.7 mg/ml-3.2 mg/ml | 3 mg/ml-20 mg/ml |
| Benzyl alcohol | 9.0 mg/ml-11.0 mg/ml | 7 mg/ml-35 mg/ml |
| Chlorobutanol | 3.0 mg/ml-5 mg/ml | 4 mg/ml-16 mg/ml |

Higher concentrations were tested than those commonly used in drug products, in order to reflect what the concentration might be in the excipient solution or in the mixture of DS and excipient solution: the concentration in the latter two will always be higher than the final concentration in the drug product.

The study was performed by adding an aqueous solution containing a surfactant to an aqueous solution containing a preservative. The pH was adjusted to 6.0, the solutions were mixed and the solutions evaluated immediately.

All of the solutions were visually inspected according to Assay I. All interactions between preservatives and surfactants were seen in the form of a suspension of white precipitates. The clarity of the solutions was compared with NTU standards 3, 6, 18, 30, 100 and 200. A clarity of up to 18 NTU corresponds to an almost clear solution and would be acceptable during a typical manufacturing process. 30 NTU corresponds to an unclear solution and would not be acceptable during a typical manufacturing process.

Interactions between Phenol and Surfactants

Table 4 provides the results for solutions containing one of five concentrations of phenol and either Polysorbate 20 (0.25 mg/ml), Polysorbate 80 (0.25 mg/ml) or Poloxamer 188 (1.0 mg/ml). No difference in clarity was observed between the initial solutions and the solutions that had been adjusted to pH 6.0.

TABLE 4
Clarity (NTU) of Phenol solutions with surfactants

| | Surfactant | Phenol concentration (mg/ml) | | | | |
|---|---|---|---|---|---|---|
| Surfactant | concentration | 16 mg/ml | 10 mg/ml | 8 mg/ml | 6 mg/ml | 4 mg/ml |
| Polysorbate | 0.25 mg/ml | 100 NTU | >200 NTU | 200 NTU | 100 NTU | 0 NTU |
| 20 | | | | | | |
| Polysorbate | 0.25 mg/ml | 100 NTU | >200 NTU | 200 NTU | 18 NTU | 0 NTU |
| 80 | | | | | | |
| Poloxamer | 1.00 mg/ml | >200 NTU | 100 NTU | 0 NTU | 0 NTU | 0 NTU |
| 188 | | | | | | |

The results show that interactions between Phenol and either PS20 or PS80 began to occur when the Phenol concentration was at least 6 mg/ml. In the case of 1.0 mg/ml PLX188, interactions began to occur at 10 mg/ml Phenol. The results show that for PS 20 unacceptable interactions are seen at 6 mg/ml phenol. For PS80 unacceptable interactions are seen at 8 mg/ml while for Poloxamer 188 unacceptable interactions are seen at 10 mg/ml phenol and above.

Interactions Between m-Cresol and Surfactants

Table 5 provides the results for solutions containing one of five concentrations of m-Cresol and either PS20 (0.25 mg/ml), PS80 (0.25 mg/ml) or PLX188 (1.0 mg/ml). No difference in clarity was observed between the initial solutions and the solutions that had been adjusted to pH 6.0.

TABLE 5
Clarity (NTU) of m-Cresol solutions with surfactants

| | Surfactant | m-Cresol concentration (mg/ml) | | | | |
|---|---|---|---|---|---|---|
| Surfactant | concentration | 20 mg/ml | 15 mg/ml | 10 mg/ml | 5 mg/ml | 3 mg/ml |
| Polysorbate | 0.25 mg/ml | 200 NTU | 100 NTU | 100 NTU | 200 NTU | 100 NTU |
| 20 | | | | | | |
| Polysorbate | 0.25 mg/ml | 200 NTU | 100 NTU | 100 NTU | 200 NTU | 18 NTU |
| 80 | | | | | | |
| Poloxamer | 1.00 mg/ml | >200 NTU | >200 NTU | >200 NTU | 3 NTU | 0 NTU |
| 188 | | | | | | |

The results show that interactions between m-cresol and either PS20 or PS80 occurred when the m-Cresol concentration was 3 mg/ml. In the case of PLX188, interactions were seen at 10 mg/ml m-Cresol. For PS20 unacceptable interactions occurred at all tested concentrations of m-cresol. For PS 80 unacceptable interaction occurred at 5 mg/ml m-cresol and for PLX 188 unacceptable interactions occurred at 10 mg/ml m-cresol.

### Interactions Between Benzyl Alcohol and Surfactants

Table 6 provides the results for solutions containing one of five concentrations of Benzyl alcohol and either PS20 (0.25 mg/ml), PS80 (0.25 mg/ml) or PLX188 (1.0 mg/ml). No difference in clarity was observed between the initial solutions and the solutions that had been adjusted to pH 6.0.

TABLE 6
Clarity (NTU) of Benzyl alcohol solutions with surfactants

| | Surfactant | Benzyl alcohol concentration (mg/ml) | | | | |
|---|---|---|---|---|---|---|
| Surfactant | concentration | 35 mg/ml | 20 mg/ml | 15 mg/ml | 10 mg/ml | 7 mg/ml |
| Polysorbate | 0.25 mg/ml | >200 NTU | 0 NTU | 0 NTU | 0 NTU | 0 NTU |
| 20 | | | | | | |
| Polysorbate | 0.25 mg/ml | >200 NTU | 0 NTU | 0 NTU | 0 NTU | 0 NTU |
| 80 | | | | | | |
| Poloxamer | 1.00 mg/ml | 3 NTU | 0 NTU | 0 NTU | 0 NTU | 0 NTU |
| 188 | | | | | | |

The results show that interactions occur between Benzyl alcohol and either PS20 or PS80 when the Benzyl alcohol concentration is 35 mg/ml, with the Clarity scale rising to >200 NTU at 35 mg/ml Benzyl alcohol. In the case of PLX188, only a slight interaction (3 NTU) was observed with 35 mg/ml Benzyl alcohol. For PS20 and PS80 unacceptable interaction occurred at 35 mg/ml benzyl alcohol.

### Interactions Between Chlorobutanol and Surfactants

Table 7 provides the results for solutions containing one of five Chlorobutanol concentrations and either PS20 (0.25 mg/ml), PS80 (0.25 mg/ml) or PLX188 (1.0 mg/ml). No difference in clarity was observed between the initial solutions and the solutions that had been adjusted to pH 6.0.

TABLE 7
Clarity (NTU) of Chlorobutanol solutions with surfactants

| | | Chlorobutanol concentration (mg/ml) | | | | |
|---|---|---|---|---|---|---|
| | Surfactant | 7.5 | 6.5 | 5.5 | 5.0 | 4.0 |
| Surfactant | concentration | mg/ml | mg/ml | mg/ml | mg/ml | mg/ml |
| Polysorbate | 0.25 mg/ml | 18 NTU | 0 NTU | 0 NTU | 0 NTU | 0 NTU |
| 20 | | | | | | |
| Polysorbate | 0.25 mg/ml | 3 NTU | 0 NTU | 0 NTU | 0 NTU | 0 NTU |
| 80 | | | | | | |
| Poloxamer | 1.00 mg/ml | 0 NTU | 0 NTU | 0 NTU | 0 NTU | 0 NTU |
| 188 | | | | | | |

The results show that interactions between PS20 or PS80 and Chlorobutanol begin to occur at 7.5 mg/ml Chlorobutanol, with the Clarity scale rising from 0 NTU to 18 NTU and 3 NTU between 6.5 mg/ml and 7.5 mg/ml Chlorobutanol for PS20 and PS80, respectively. No interaction between Chlorobutanol and PLX188 was observed within the tested Chlorobutanol concentration range. For all tested surfactants, no unacceptable interactions were observed for Chlorobutanol.

These combined results show that there is some degree of interaction between all combinations of preservatives and surfactants tested within this study at high concentrations; with the exception of the Chlorobutanol and PLX188 combination, in which case no interactions were observed within the tested concentration range.

Based on the result from the interactions, it is evident that special care should be taken when mixing preservatives and surfactants.

The concentration of benzyl alcohol should be less than 35 mg/ml in the solution to which Polysorbate 20 or Polysorbate 80 is added. The concentration of benzyl alcohol may be greater than 35 mg/ml in the solution to which Poloxamer 188 is added.

The concentration of chlorobutanol may be as great as 7.5 mg/ml in the solution to which Polysorbate 20, Polysorbate 80 or Poloxamer 188 is added.

The concentration of m-cresol should be less than 3 mg/ml in the solution to which Polysorbate 20 is added. The concentration of m-cresol should not exceed 5 mg/ml in the solution to which Polysorbate 80 is added. The concentration of m-cresol should be less than 10 mg/ml in the solution to which Poloxamer 188 is added.

The concentration of phenol should be less than 6 mg/ml in the solution to which Polysorbate 20 is added. The concentration of phenol should be less than 8 mg/ml in a solution to which Polysorbate 80 is added. The concentration of phenol should be less than 10 mg/ml in the solution to which Poloxamer 188 is added.

The quality of the final drug product would be affected if a higher concentration of preservative were used.

### Example 3: Preservatives and Surfactants can Interact to Form Unwanted Opalescence

As demonstrated in this as well as the previous example, preservatives and surfactants can interact to form unwanted opalescence in a formulation. Compositions containing a variety of concentrations of phenol and polysorbate 80 were tested. The purpose of the experiment was to determine the concentrations at which interactions between phenol and polysorbate 80 start to occur.

Aqueous solutions containing different concentrations of phenol and polysorbate 80 were prepared in a 96 well plate. The concentrations fell within the ranges provided in table 8 and are shown in table 9. Where the final drug product is to contain 100 mg/ml concizumab, the concentration of phenol in the excipient solution may be, for example, 10 times higher than the concentration of phenol in the final DP. So this reflects a final Phenol concentration of 0-5.5 mg/ml.

TABLE 8
Concentration ranges

| Component | Tested concentration range |
|---|---|
| Phenol | 0 mg/ml-55 mg/ml |
| Polysorbate 80 | 0.01 mg/ml-0.35 mg/ml |

The solutions were prepared and subsequently analysed using a ClaroSTAR plate reader (Assay Ill). The results are given as changes in light intensity, which can be used to determine interactions between the components in the solutions. The results are provided in table 9. A two-fold change in light intensity, compared to the solution without phenol, is indicative of interactions occurring in the solution.

TABLE 9
ClarioSTAR results

| Polysorbate 80 | | | | | | | | | | | | |
|---|---|---|---|---|---|---|---|---|---|---|---|---|
| concentration | Phenol concentrations (mg/ml) | | | | | | | | | | | |
| (mg/ml) | 0 | 2.5 | 5 | 10 | 15 | 20 | 30 | 35 | 40 | 45 | 50 | 55 |
| 0.01 | 0.044 | 0.047 | 0.049 | 0.066 | 0.077 | 0.081 | 0.086 | 0.086 | 0.081 | 0.082 | 0.074 | 0.079 |
| 0.03 | 0.065 | 0.052 | 0.054 | 0.095 | 0.11 | 0.122 | 0.118 | 0.103 | 0.107 | 0.113 | 0.109 | 0.108 |
| 0.1 | 0.05 | 0.056 | 0.053 | 0.107 | 0.12 | 0.159 | 0.173 | 0.162 | 0.174 | 0.179 | 0.189 | 0.201 |
| 0.15 | 0.051 | 0.054 | 0.06 | 0.148 | 0.223 | 0.162 | 0.207 | 0.217 | 0.241 | 0.248 | 0.262 | 0.295 |
| 0.2 | 0.05 | 0.053 | 0.066 | 0.242 | 0.209 | 0.228 | 0.242 | 0.305 | 0.301 | 0.315 | 0.42 | 0.366 |
| 0.25 | 0.05 | 0.052 | 0.058 | 0.326 | 0.402 | 0.494 | 0.39 | 0.371 | 0.387 | 0.396 | 0.436 | 0.471 |
| 0.3 | 0.048 | 0.05 | 0.059 | 0.451 | 0.494 | 0.562 | 0.518 | 0.496 | 0.482 | 0.531 | 0.551 | 0.612 |
| 0.35 | 0.049 | 0.051 | 0.064 | 0.527 | 0.509 | 0.556 | 0.61 | 0.545 | 0.596 | 0.604 | 0.671 | 0.755 |

The results indicate that interactions between phenol and polysorbate 80 start to occur when the concentration of phenol is between 5 and 10 mg/ml and the concentration of polysorbate 80 is higher than 0.1 mg/ml. This is in agreement with the results presented in example 1. The results show that even at the lower concentration of 0.03 mg/ml polysorbate 80, interactions occur when the concentration of phenol is more than 10 mg/ml. These results suggest that the concentration of phenol should generally not exceed 10 mg/ml in the solution to which Polysorbate 80 is added, as this would cause unwanted opalescence during manufacture. The quality of the final drug product might be affected if a higher concentration is used. Where the concentration of polysorbate 80 is less than 0.03 mg/ml, a concentration of up to 15 mg/ml phenol may be acceptable.

### Example 4: pH Adjustment Readily Results in the Formation of Protein Impurities

The purpose of this experiment was to determine the stability of concizumab in compositions where pH was adjusted to pH 5.6.

The formulation comprising concizumab used was an aqueous solution (drug substance) containing concizumab (100 mg/ml or 10 mg/ml), L-Arginine-HCl (5.27 mg/ml), L-Histidine (5.12 mg/ml), Sodium Chloride (1.46 mg/ml), Sucrose (51.3 mg/ml), Phenol (3.5 mg/ml), and Polysorbate 80 (0.01 mg/ml) and had a pH of 6.0.

Two methods of adjusting the pH of the formulation comprising concizumab were compared. In Method 1, 2N hydrochloric acid was added to the formulation comprising concizumab, to adjust its pH directly. Method 2 made use of a preformulated drug substance having a lower pH and an excipient solution, to which 2N hydrochloric acid was added for pH adjustment before drug substance was added to the excipient solution.

For Method 1, an excipient solution was prepared by dissolving L-Arginine-HCl, L-Histidine, Sodium Chloride, Sucrose and Phenol in Water For Injection and adjusting the solution to pH 6. An initial amount of hydrochloric acid was added to the excipient solution, followed by preformulated drug substance. The pH of the mixture was then adjusted to pH 5.6, using 2N hydrochloric acid.

For Method 2, an excipient solution was prepared by dissolving L-Arginine-HCl, L-Histidine, Sodium Chloride, Sucrose and Phenol in Water For Injection and then adjusting the solution to pH 5.6 by adding 2N hydrochloric acid. Buffer exchanged, preformulated drug substance (DS) having pH 5.6 was then added to the excipient solution. 2N hydrochloric acid was not added directly to the formulation comprising concizumab or to the drug product (DP).

The chemical stability of the compositions prepared was determined by measuring the formation of HMWP, as described in Assay II. The results are shown in table 10.

TABLE 10
Results for pH adjustment

| | pH | | HMWP % | | |
|---|---|---|---|---|---|
| | adjustment | | | | Increase |
| | directly | Concizumab | | | during |
| Experi- | into | concentration | Drug | Drug | formulation |
| ment | DS/DP | (mg/ml) | Substance | Product | process |
| 1 | No | 100 | 0.7 | 1.1 | 0.4 |
| 2 | No | 10 | 0.7 | 0.7 | None |
| 3 | Yes | 100 | 0.6 | 2.9 | 2.3 |
| 4 | Yes | 10 | 0.6 | 3.3 | 2.7 |

The results show that interactions occur between acid (HCl) and antibody when an acid is added directly to a composition containing the antibody.

Adjusting pH by adding acid directly to the composition comprising concizumab, readily resulted in the formation of unwanted protein impurities such as high molecular weight protein (HMWP). The addition of acid (HCl) to either the drug substance (DS) or the drug product (DP) resulted in an unacceptable increase in the percentage of HMWP present in the final pharmaceutical formulation (DP). In contrast, the increase in % HMWP was minor and at an acceptable level when pH was adjusted using method 2 (which is part of the manufacturing method 1 described in example 6).

In any manufacturing process, an increase of 2.3% or 2.7% HMWP is not considered acceptable. In order not to affect the quality of the final drug product (i.e., pharmaceutical formulation), concentrated acid should not be added directly to a composition containing concizumab.

### Example 5: Effect of Polysorbate 80 Concentration on Physical Stability

A surfactant such as polysorbate 80 is included in the concizumab drug product to prevent surface adsorption to containers and production materials and to stabilise the protein against protein aggregation. The stability of the composition might be improved by increasing the content of polysorbate 80.
The batches tested contained concizumab (10 mg/ml and 100 mg/ml) as specified in table 11, L-Arginine-HCl (5.27 mg/ml), L-Histidine (5.12 mg/ml), Sodium Chloride (1.46 mg/ml), Sucrose (51.3 mg/ml, Phenol (3.5 mg/ml), and Polysorbate 80 (0.01 mg/ml to 0.3 mg/ml) as specified in table 11 at pH 6.0 in aqueous solution. Tested compositions were prepared according to the currently claimed manufacturing method (embodied by manufacturing method 1 in example 6).
A drug substance (DS) comprising concizumab and having a pH of 6.0, was prepared using the downstream process described in WO2009/138484. The concentration of concizumab in the drug substance was 123 mg/ml. The surfactant-free excipient solutions were prepared by dissolving L-Arginine HCl, L Histidine, Sucrose, Sodium Chloride and Phenol in Water For Injection (75% of final concizumab drug product (DP) volume for 10 mg/ml, 10% of final concizumab DP volume for 100 mg/ml). The pH was adjusted to 6.0 using 2N Hydrochloric acid and/or 2N Sodium Hydroxide and the solution is homogenized. Concizumab drug substance was added to the solution and homogenised. Polysorbate 80 was added and the mixture was homogenised. These solutions contained 0.01-0.36 mg/ml Polysorbate 80 and 4.2 mg/ml Phenol for a 10 mg/ml concizumab DP and 0.01-0.32 mg/ml polysorbate 80 and 3.8 mg/ml Phenol for a 100 mg/ml concizumab DP. Water For Injection was added to 100% of the final volume. The formulation was mixed and homogenized before being sterile filtered and filled in 1.5 ml cartridges.

TABLE 11
Tested compositions

| Composition no. | Concizumab (mg/mL) | Polysorbate 80 (mg/mL) |
|---|---|---|
| 1 | 10 | 0.01 |
| 2 | 10 | 0.05 |
| 3 | 10 | 0.1 |
| 4 | 10 | 0.2 |
| 5 | 10 | 0.3 |
| 6 | 100 | 0.01 |
| 7 | 100 | 0.05 |
| 8 | 100 | 0.1 |
| 9 | 100 | 0.2 |
| 10 | 100 | 0.3 |

The chemical stability of the prepared batches was determined by measuring the formation of HMWP as described in Assay II. The results are shown in table 12.

TABLE 12
Results for HMWP(%)

| | Concizumab | Polysorbate 80 | |
|---|---|---|---|
| Batch no. | (mg/ml) | (mg/ml) | HMWP (%) |
| 1 | 10 | 0.01 | 0.4 |
| 2 | 10 | 0.05 | 0.4 |
| 3 | 10 | 0.1 | 0.4 |
| 4 | 10 | 0.2 | 0.3 |
| 5 | 10 | 0.3 | 0.3 |
| 6 | 100 | 0.01 | 0.6 |
| 7 | 100 | 0.05 | 0.6 |
| 8 | 100 | 0.1 | 0.5 |
| 9 | 100 | 0.2 | 0.5 |
| 19 | 100 | 0.3 | 0.5 |

The results presented in table 12 show that the level of HMWP is comparable when different concentrations of polysorbate 80 are present in the composition, i.e. the composition is chemically stable regardless of the polysorbate 80 concentration.
The batchess containing concizumab were also tested for physical stability using Micro Flow Imaging (MFI) as described in Assay IV. The results are an average of 3 measurements. This is to measure the amount of sub-visible particles in the batchess. Results are given in table 13. The results show that the number of subvisible particles decreases when the concentration of Polysorbate 80 is 0.1 mg/ml, preferably at least 0.2 mg/ml.

TABLE 13
Results for subvisible particle

| | Concizumab | Polysorbate | Mean particles (#/mL) | | |
|---|---|---|---|---|---|
| Batch no. | (mg/ml) | 80 (mg/ml) | >5 μm | >10 μm | >25 μm |
| 1 | 10 | 0.01 | 6129 | 1845 | 108 |
| 2 | 10 | 0.05 | 2187 | 693 | 50 |
| 3 | 10 | 0.1 | 657 | 174 | 19 |
| 4 | 10 | 0.2 | 115 | 16 | 3 |
| 5 | 10 | 0.3 | 19 | 2 | 1 |
| 6 | 100 | 0.01 | 53564 | 15911 | 451 |
| 7 | 100 | 0.05 | 828 | 322 | 65 |
| 8 | 100 | 0.1 | 200 | 72 | 7 |
| 9 | 100 | 0.2 | 87 | 41 | 13 |
| 10 | 100 | 0.3 | 38 | 11 | 1 |

The results presented in table 12 and table 13 show that when polysorbate 80 is present at a concentration above 0.1 mg/ml, and preferably above 0.2 mg/ml, in the batches of concizumab drug product, a similar amount of HMWP is formed but fewer subvisible particles are formed, i.e. the composition is chemically equally stable but physically more stable.

### Example 6: Manufacturing Methods

The purpose of this study was to determine the effects of different manufacturing methods and the order of addition of ingredients on the final quality of a concizumab drug product (DP). The manufacturing methods tested were used to produce formulations containing concizumab (10 mg/ml and 100 mg/ml), L-Arginine-HCl (5.27 mg/ml), L-Histidine (5.12 mg/ml), Sodium Chloride (1.46 mg/ml), Sucrose (51.3 mg/ml), Polysorbate 80 (0.25 mg/ml) and Phenol (3.5 mg/ml), and a pH of 6.0 in aqueous solution.

Seven different manufacturing methods were tested. Manufacturing method 1 embodies the currently claimed manufacturing method. Formulations were evaluated by visual inspection and assessed by analysing for the content of HMWP in the concizumab drug substance prior to formulation and the final concizumab drug products.

### Manufacturing Method 1:

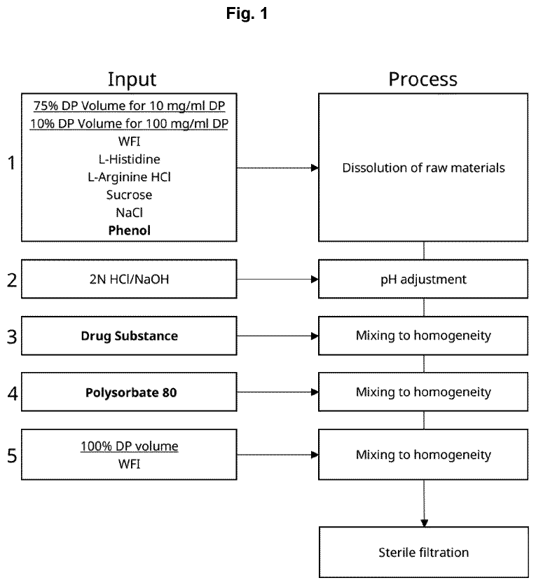

A flow diagram for this manufacturing method can be found in FIG. 1. A drug substance (DS) comprising concizumab and having a pH of 6.0 was prepared using the downstream process described in WO2009/138484.
1. L-Arginine HCl, L Histidine, Sucrose, Sodium Chloride and Phenol was dissolved in Water For Injection (10% of final volume for 100 concizumab mg/ml DP and 75% of the final volume for 10 concizumab mg/ml DP). This solution contained 4.7 mg/ml Phenol for a 10 mg/ml concizumab DP and 35 mg/ml Phenol for a 100 mg/ml concizumab DP).
2. pH was adjusted to 6.0 using 2N Hydrochloric acid and/or 2N Sodium Hydroxide and the solution is homogenised. pH was adjusted to 6.0 using 2N Hydrochloric acid and/or 2N Sodium Hydroxide and the solution was homogenized.
3. Concizumab drug substance (115 mg/ml) was added to the solution and homogenised. The volume of this solution was 84% of the final DP volume for a 10 mg/ml concizumab DP and 97% of the final DP volume for a 100 mg/ml concizumab DP). The volume of this solution was 84% of the final DP volume for a 10 mg/ml concizumab DP and 97% of the final DP volume for a 100 mg/ml concizumab DP.
4. Polysorbate 80 (30 mg/ml stock solution) was added and homogenised. This solution contained 0.32 mg/ml Polysorbate 80 and 4.5 mg/ml Phenol for 10 mg/ml DP and 0.26 mg/ml polysorbate 80 and 3.6 mg/ml Phenol for 100 mg/ml DP.
5. Water For Injection was added to 100% of the final volume. The formulation was homogenized before being sterile filtered and filled into cartridges.

### Manufacturing Method 2:

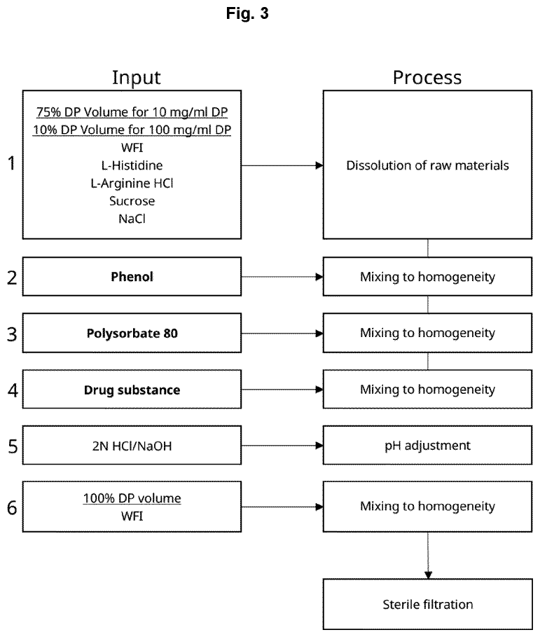

A flow diagram for this manufacturing method can be found in FIG. 3.
1. L-Arginine, L Histidine, Sucrose, Sodium Chloride and Phenol was dissolved in Water For Injection (10% of the final volume for 100 mg/ml concizumab DP and 75% of the final volume for 10 mg/ml concizumab DP).
2. pH was adjusted to 6.0 using 2N Hydrochloric acid and/or 2N Sodium Hydroxide and the solution was homogenized.
3. Polysorbate 80 was added and dissolved. This solution contained 0.36 mg/ml Polysorbate 80 and 5.0 mg/ml Phenol for 10 mg/ml concizumab DP and 2.5 mg/ml polysorbate 80 and 35 mg/ml Phenol for 100 mg/ml concizumab DP.
4. Concizumab drug substance (115 mg/ml) was added to the solution and homogenised.
5. Water For Injection was added to 100% of the final volume. The formulation was homogenized before being sterile filtered and filled into cartridges.

### Manufacturing Method 3

A flow diagram for this manufacturing method can be found in FIG. 3.
1. L-Arginine, L Histidine, Sucrose and Sodium Chloride was dissolved in Water For Injection (10% of the final volume for 100 mg/ml concizumab DP and 75% of the final volume for 10 mg/ml concizumab DP).
2. Phenol was added and dissolved.
3. Polysorbate 80 was added and dissolved. This solution contained 0.36 mg/ml Polysorbate 80 and 5.0 mg/ml Phenol for 10 mg/ml DP and 2.5 mg/ml polysorbate 80 and 35 mg/ml Phenol for 100 mg/ml DP.
4. Concizumab drug substance (125 mg/ml) was added to the solution and homogenised.
5. pH was adjusted to 6.0 using 2N Hydrochloric acid and/or 2N Sodium Hydroxide and the solution was homogenized.
6. Water For Injection was added to 100% of the final volume. The formulations were homogenized before being sterile filtered and filled into cartridges.

### Manufacturing Method 4:

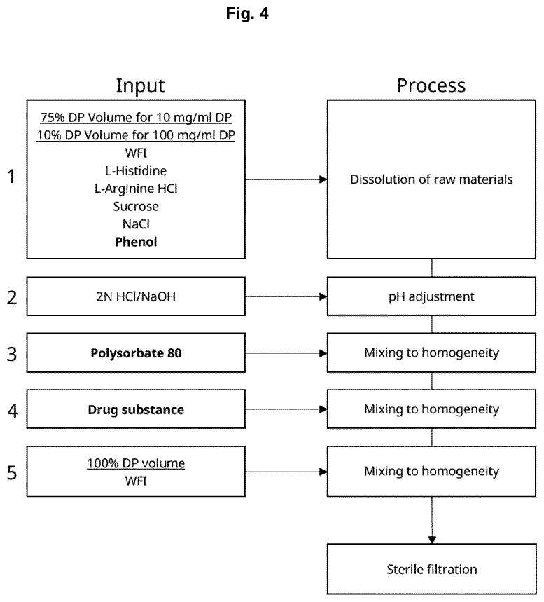

A flow diagram for this manufacturing method can be found in FIG. 4.
1. L-Arginine, L Histidine, Sucrose, Sodium Chloride and Polysorbate 80 was dissolved in Water For Injection (10% of the final volume for 100 mg/ml concizumab DP and 75% if the final volume for 10 mg/ml concizumab DP).
2. pH was adjusted to 6.0 using 2N Hydrochloric acid and/or 2N Sodium Hydroxide and the solution was homogenized.
3. Drug substance (115 mg/ml) was added to the solution and homogenised.
4. Phenol was added and dissolved. This solution contained 0.32 mg/ml Polysorbate 80 and 4.5 mg/ml Phenol for 10 mg/ml concizumab DP and 0.26 mg/ml polysorbate 80 and 3.6 mg/ml Phenol for 100 mg/ml concizumab DP.
5. Water For Injection was added to 100% of the final volume. The formulation was homogenized before being sterile filtered and filled into cartridges.

### Manufacturing Method 5

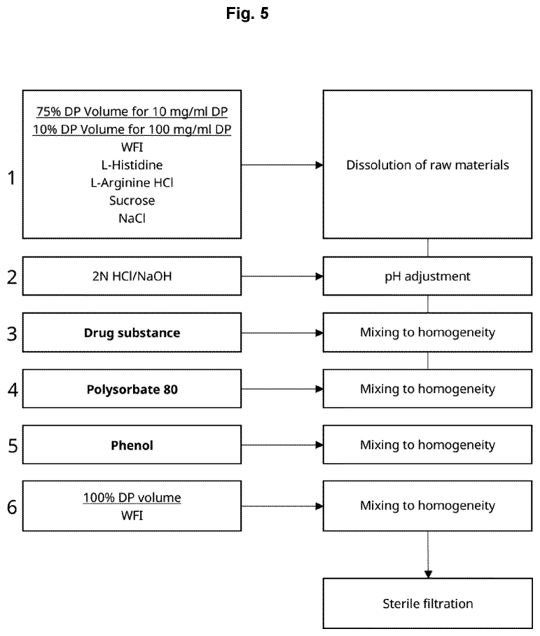

A flow diagram for this manufacturing method can be found in FIG. 5.
1. L-Arginine, L Histidine, Sucrose and Sodium Chloride was dissolved in Water For Injection (10% of the final volume for 100 mg/ml concizumab DP and 75% of the final volume for 10 mg/ml concizumab DP).
2. pH was adjusted to 6.0 using 2N Hydrochloric acid and/or 2N Sodium Hydroxide and the solution was homogenized.
3. Concizumab drug substance (125 mg/ml) was added to the solution and homogenised.
4. Polysorbate 80 was added and dissolved.
5. Phenol was added and dissolved. This solution contained 0.32 mg/ml Polysorbate 80 and 4.5 mg/ml Phenol for 10 mg/ml concizumab DP and 0.28 mg/ml polysorbate 80 and 3.9 mg/ml Phenol for 100 mg/ml concizumab DP.
6. Water For Injection was added to 100% of the final volume. The formulations were homogenized before being sterile filtered and filled into cartridges.

### Manufacturing Method 6:

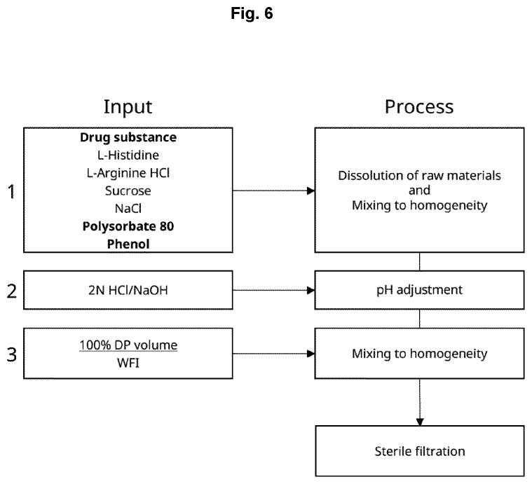

A flow diagram for this manufacturing method can be found in FIG. 6.
1. L-Arginine, L Histidine, Sucrose, Sodium Chloride, Phenol and Polysorbate 80 were dissolved in concizumab drug substance (115 mg/ml). This solution contained 2.9 mg/ml Polysorbate 80 and 40 mg/ml Phenol for 10 concizumab mg/ml DP and 0.29 mg/ml polysorbate 80 and 4.0 mg/ml Phenol for 100 mg/ml concizumab DP.
2. The pH was adjusted to 6.0 using 2N Hydrochloric acid and/or 2N Sodium Hydroxide and the solution was homogenized.
3. Water For Injection was added to 100% of the final volume. The formulations were homogenized before being sterile filtered and filled into cartridges.

### Manufacturing Method 7:

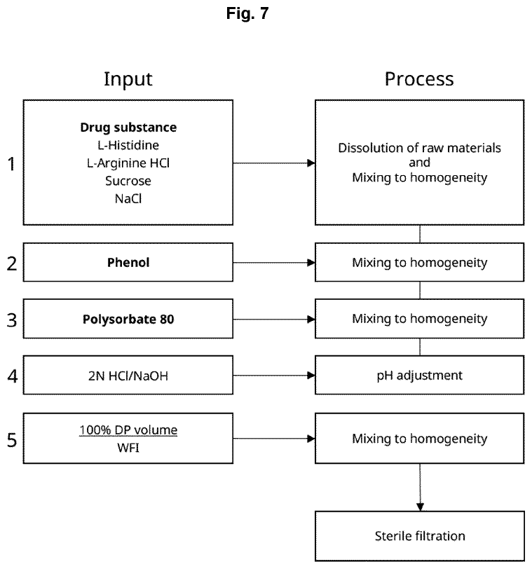

A flow diagram for this manufacturing method can be found in FIG. 7.
1. L-Arginine, L Histidine, Sucrose and Sodium Chloride were dissolved in concizumab drug substance (125 mg/ml).
2. Phenol was added and dissolved.
3. Polysorbate was added and dissolved. This solution contained 3.1 mg/ml Polysorbate 80 and 44 mg/ml Phenol for 10 concizumab mg/ml DP and 0.31 mg/ml polysorbate 80 and 4.4 mg/ml Phenol for 100 mg/ml concizumab DP
4. pH was adjusted to 6.0 using 2N Hydrochloric acid and/or 2N Sodium Hydroxide and the solution was homogenized. pH was adjusted to 6.0 using 2N Hydrochloric acid and/or 2N Sodium Hydroxide and the solution was homogenized.
5. Water For Injection was added to 100% of the final volume. The formulations are homogenized before being sterile filtered and filled into cartridges.

### Results

An overview of observations during manufacturing methods can be seen in table 14 and an overview of HMWP (%) results can be found in table 15.

TABLE 14
Observations during manufacturing methods

| Manufacturing | Concizumab | |
|---|---|---|
| method | (mg/ml) | Observations during manufacturing |
| 1 | 10 | All solutions were clear after dissolution and |
| | | homogenisation of all ingredients. |
| | 100 | All solutions were clear after dissolution and |
| | | homogenisation of all ingredients. |
| 2 | 10 | Step 3: Unwanted opalescence was formed in the |
| | | solution upon addition of Polysorbate 80, which did not |
| | | become clear after prolonged stirring. |
| | 100 | Step 3: Unwanted opalescence was formed in the |
| | | solution upon addition of Polysorbate 80, which did not |
| | | become clear after prolonged stirring. |
| 3 | 10 | Step 3: Unwanted opalescence was formed in the |
| | | solution upon addition of Polysorbate 80, which did not |
| | | become clear after prolonged stirring. |
| | 100 | Step 3: Unwanted opalescence was formed in the |
| | | solution upon addition of Polysorbate 80, which did not |
| | | become clear after prolonged stirring. Preparation was |
| | | discontinued after this step. |
| 4 | 10 | Step 4: Heavy, insoluble precipitation upon addition of |
| | | Phenol. Preparation was discontinued after this step. |
| | 100 | Step 4: Heavy, insoluble precipitation upon addition of |
| | | Phenol. Preparation was discontinued after this step. |
| 5 | 10 | Step 5: Heavy, insoluble precipitation upon addition of |
| | | Phenol. |
| | 100 | Step 5: Heavy, insoluble precipitation upon addition of |
| | | Phenol. Preparation was discontinued after this step. |
| 6 | 10 | Not tested |
| | 100 | Step 1: Heavy, insoluble precipitation. Preparation was |
| | | discontinued after this step. |
| 7 | 10 | Not tested |
| | 100 | Step 2: Heavy, insoluble precipitation upon addition of |
| | | Phenol. Preparation was discontinued after this step. |

TABLE 15
Results for HMWP (%)

| | | HMWP (%) | | |
|---|---|---|---|---|
| | | | | Increase |
| | | | | during |
| Manufacturing | Concizumab | Drug | Drug | formulation |
| method | (mg/ml) | Substance | Product | process |
| 1 | 10 | 0.6 | 0.7 | 0.1 |
| | 100 | 0.6 | 0.6 | 0.0 |
| 2 | 10 | 0.6 | 0.7 | 0.1 |
| | 100 | 0.6 | Note A | N/A |
| 3 | 10 | 0.6 | 3.8 | 3.2 |
| | 100 | 0.6 | Note A | N/A |
| 4 | 10 | 0.6 | Note B | N/A |
| | 100 | 0.6 | Note B | N/A |
| 5 | 10 | 0.6 | 2.5 | 1.9 |
| | 100 | 0.6 | Note B | N/A |
| 6 | 10 | — | Note C | N/A |
| | 100 | 0.6 | Note B | N/A |
| 7 | 10 | — | Note C | N/A |
| | 100 | 0.6 | Note B | N/A |

Note A:
Discontinued due to unwanted opalescence after addition of PS80
Note B:
Preparation discontinued after addition of Phenol, due to heavy precipitation.
Note C:
Not tested

In manufacturing method 1, which embodies the currently claimed manufacturing method, all solutions in each manufacturing step were clear after dissolution and homogenisation, for both concizumab 10 mg/ml drug product and 100 mg/ml drug product. The increase in % HMWP was minor and at an acceptable level.

In manufacturing process 2 and 3, it was demonstrated that adding polysorbate 80 and phenol before the addition of concizumab drug substance leads to solutions within the range of 0.28-2.5 mg/ml polysorbate 80 and 5.0 and 35 mg/ml Phenol and causes unwanted opalescence, which is not acceptable during a typical manufacturing process. In manufacturing method 3, the pH in the 10 mg/ml concizumab formulation was adjusted by addition of 2N HCl, after addition of concizumab drug substance. This caused an increase in HMWP of 3.2%, which is not considered acceptable.

Manufacturing methods 4-7 demonstrated, that addition of phenol after, or at the same time as the concizumab drug substance causes heavy, insoluble precipitation. The heavy precipitation makes it difficult to filter the solution, and therefore the majority of formulations within manufacturing methods 4-7 were discontinued after addition of phenol. In manufacturing method 5, the 10 mg/ml concizumab formulation was completed. In this formulation an increase in HMWP of 1.9% was observed, which is not considered acceptable.

### Example 7: Different Combinations of Preservatives and Surfactants

The compositions tested contained concizumab (10 mg/ml), L-Arginine-HCl (5.27 mg/ml), L-Histidine (5.12 mg/ml), Sodium Chloride (1.46 mg/ml), Sucrose (51.3 mg/ml, preservative and surfactant as specified in table 16 at pH 6.0 in aqueous solution. Tested compositions were prepared according to the claimed manufacturing method (Manufacturing method 1 in example 6).

TABLE 16
Compositions tested

| | | Concentration | | Concentration |
|---|---|---|---|---|
| Batch no. | Preservative | (mg/ml) | Surfactant | (mg/ml) |
| 1 | Phenol | 4.0 | Polysorbate 20 | 0.25 |
| 2 | Phenol | 4.0 | Polysorbate 80 | 0.25 |
| 3 | Phenol | 4.0 | Poloxymer 188 | 1.00 |
| 4 | M-cresol | 3.0 | Polysorbate 20 | 0.25 |
| 5 | M-cresol | 3.0 | Polysorbate 80 | 0.25 |
| 6 | M-cresol | 3.0 | Poloxymer 188 | 1.00 |
| 7 | Chlorobutanol | 5.0 | Polysorbate 20 | 0.25 |
| 8 | Chlorobutanol | 5.0 | Polysorbate 80 | 0.25 |
| 9 | Chlorobutanol | 5.0 | Poloxymer 188 | 1.00 |
| 10 | Benzyl alcohol | 10.0 | Polysorbate 20 | 0.25 |
| 11 | Benzyl alcohol | 10.0 | Polysorbate 80 | 0.25 |
| 12 | Benzyl alcohol | 10.0 | Poloxymer 188 | 1.00 |
| 13 (reference) | Phenol | 3.5 | Polysorbate 80 | 0.25 |

A drug substance (DS) comprising concizumab and having a pH of 6.0, was prepared using the downstream process described in WO2009/138484. The concentration of concizumab in this drug substances was 139 mg/ml. The surfactant-free excipient solutions were prepared by dissolving L-Arginine HCl, L Histidine, Sucrose, Sodium Chloride and Preservative in Water For Injection (75% of final concizumab 10 mg/ml DP volume). The pH is adjusted to 6.0 using 2N Hydrochloric acid and/or 2N Sodium Hydroxide and the solution is homogenized. Concizumab drug substance was added to the solution and homogenised. Surfactant was added and homogenised. Water For Injection is added to 100% of the final volume. The formulation was mixed and homogenized before being sterile filtered and filled in 1.5 ml cartridges.

During preparation, unwanted opalescence was observed for batches 4 and 5 (m-cresol in combination with Polysorbate 20 and Polysorbate 80, respectively) in solution comprising excipient solution and drug substance after surfactant was added. This is in agreement with the observation in example 2, table 5.

Batches were stored at 30° C. and 40° C. and their stability was monitored for 4 weeks. The chemical stability of the prepared compositions was determined by measuring the formation of HMWP, as described in Assay II. The results are shown in table 17 and table 18, respectively.

TABLE 17
HMWP (%) result during storage at 30° C.

| Batch | | Conc. | | Conc. | HMWP (%) | | |
|---|---|---|---|---|---|---|---|
| no. | Preservative | (mg/ml) | Surfactant | (mg/ml) | 0 weeks | 2 weeks | 4 weeks |
| 1 | Phenol | 4.0 | Polysorbate 20 | 0.25 | 0.7 | 0.7 | 0.7 |
| 2 | Phenol | 4.0 | Polysorbate 80 | 0.25 | 0.7 | 0.7 | 0.7 |
| 3 | Phenol | 4.0 | Poloxymer 188 | 1.00 | 0.6 | 0.6 | 0.7 |
| 4 | M-cresol | 3.0 | Polysorbate 20 | 0.25 | 0.7 | Not tested | Not tested |
| 5 | M-cresol | 3.0 | Polysorbate 80 | 0.25 | 0.8 | Not tested | Not tested |
| 6 | M-cresol | 3.0 | Poloxymer 188 | 1.00 | 0.7 | 0.6 | 0.7 |
| 7 | Chlorobutanol | 5.0 | Polysorbate 20 | 0.25 | 0.6 | 0.7 | 0.6 |
| 8 | Chlorobutanol | 5.0 | Polysorbate 80 | 0.25 | 0.6 | 0.6 | 0.6 |
| 9 | Chlorobutanol | 5.0 | Poloxymer 188 | 1.00 | 0.6 | 0.6 | 0.6 |
| 10 | Benzyl | 10.0 | Polysorbate 20 | 0.25 | 0.6 | 0.6 | 0.7 |
| | alcohol | | | | | | |
| 11 | Benzyl | 10.0 | Polysorbate 80 | 0.25 | 0.6 | 0.6 | 0.7 |
| | alcohol | | | | | | |
| 12 | Benzyl | 10.0 | Poloxymer 188 | 1.00 | 0.6 | 0.6 | 0.7 |
| | alcohol | | | | | | |
| 13 | Phenol | 3.5 | Polysorbate 80 | 0.25 | 0.7 | 0.7 | 0.8 |
| (reference) | | | | | | | |

TABLE 18
HMWP (%) result during storage at 40° C.

| | | Conc. | | Conc. | HMWP (%) | | |
|---|---|---|---|---|---|---|---|
| Batch no. | Preservative | (mg/ml) | Surfactant | (mg/ml) | 0 weeks | 2 weeks | 4 weeks |
| 1 | Phenol | 4.0 | Polysorbate 20 | 0.25 | 0.7 | 1.2 | 1.9 |
| 2 | Phenol | 4.0 | Polysorbate 80 | 0.25 | 0.7 | 1.2 | 1.9 |
| 3 | Phenol | 4.0 | Poloxymer 188 | 1.00 | 0.6 | 1.2 | 1.9 |
| 4 | M-cresol | 3.0 | Polysorbate 20 | 0.25 | 0.7 | 1.7 | 3.1 |
| 5 | M-cresol | 3.0 | Polysorbate 80 | 0.25 | 0.8 | 1.6 | 2.9 |
| 6 | M-cresol | 3.0 | Poloxymer 188 | 1.00 | 0.7 | 1.4 | 2.4 |
| 7 | Chlorobutanol | 5.0 | Polysorbate 20 | 0.25 | 0.6 | 0.6 | 1.5 |
| 8 | Chlorobutanol | 5.0 | Polysorbate 80 | 0.25 | 0.6 | 1.0 | 1.5 |
| 9 | Chlorobutanol | 5.0 | Poloxymer 188 | 1.00 | 0.6 | 1.0 | 1.4 |
| 10 | Benzyl | 10.0 | Polysorbate 20 | 0.25 | 0.6 | 1.4 | 2.4 |
| | alcohol | | | | | | |
| 11 | Benzyl | 10.0 | Polysorbate 80 | 0.25 | 0.6 | 1.4 | 2.3 |
| | alcohol | | | | | | |
| 12 | Benzyl | 10.0 | Poloxymer 188 | 1.00 | 0.6 | 1.4 | 2.1 |
| | alcohol | | | | | | |
| 13 | Phenol | 3.5 | Polysorbate 80 | 0.25 | 0.7 | 1.2 | 1.8 |
| (reference) | | | | | | | |

Results show that all batches have a similar level of HMWP (%) after preparation. The results in table 17 show that the chemical stability of all batches remains constant when they are stored at 30° C. for 4 weeks. Results in table 18 show that stability trends are similar for batches prepared with preservatives phenol, chlorobutanol or benzyl alcohol, regardless of the surfactant used. Stability trends are slightly more favorable for batches prepared with chlorobutanol compared to phenol, whereas batches prepared with benzylalcohol are slightly less favourable. Furthermore batch 6, prepared with m-cresol and poloxamer 188, followed at similar trend to batches prepared with benzyl alcohol. However batches prepared with m-cresol in combination with polysorbate 20 and polysorbate 80 have a much steeper increase in HWMP (%) compared to batches prepared with phenol.

This study show it is possible to prepare concizumab 10 mg/ml prepared according to the claimed manufacturing method, using preservatives Phenol (4 mg/ml), Chlorobutanol (5 mg/ml) or Benzyl alcohol (10 mg/ml) in combination with all three surfactants Polysorbate 20 (0.25 mg/ml), Polysorbate 80 (0.25 mg/ml) and Poloxymer 188 (1.0 mg/ml). Furthermore it is possible to prepare concizumab 10 mg/ml using preservative m-cresol in combination with surfactant poloxamer 188. However, preparation of concizumab 10 mg/ml using m-cresol in combination with either polysorbate 20 or polysorbate 80 causes unwanted opalescence during production and a steep increase in HMWP (%) during storage at 40° C. for 4 weeks.

### Example 8: NN7415 (Concizumab) Phase 3 Explorer 7 and 8 (NN7415-4311 and NN7415-4307) Trials

Explorer 7

This is a prospective, multicentre, open label clinical trial with four arms. The trial aims to evaluate the effect and safety of daily, subcutaneous concizumab prophylaxis to no prophylaxis (on-demand treatment with bypassing agents) in reducing the number of bleeding episodes in adults and adolescents 12 years) with haemophilia A or B with inhibitors.

Initially, patients were assigned to randomisation or to allocation into non-randomised treatment arms, based on their treatment regimen before the trial.

The trial consists of a main part (24 or 32 weeks), an extension part (up to 136 weeks) and a safety follow-up part (7 weeks). The main part of the trial is completed for a patient when the patient has completed at least 24 weeks of participation (arm 1) or 32 weeks of participation (arms 2, 3 and 4). After completion of the main part of the trial, all patients are invited to continue in the extension part of the trial and receive treatment with concizumab for up to an additional 136 weeks.

Explorer 8:

This is a prospective, multicentre, open label clinical trial with two randomised arms and two non-randomised arms. The trial aims to evaluate the effect and safety of daily, subcutaneous concizumab prophylaxis to no prophylaxis (on-demand treatment with bypassing agents) in adults and adolescents 12 years) with haemophilia A or B without inhibitors. After screening patients are assigned to randomisation or to allocation into non-randomised treatment arms, based on their treatment regimen before the trial.

The trial consists of a main part (24 or 32 weeks), an extension part (up to 136 weeks) and a safety follow-up part (7 weeks). The main part of the trial is completed for a patient when the patient has completed 24 weeks of participation (arm 1) or 32 weeks of participation (arms 2, 3 and 4). After the main part of the trial, all patients are invited to continue in the extension part of the trial and receive treatment with concizumab for up to an additional 136 weeks.

Treatment in Both Trials (Explorer 7 and Explorer 8)

The drug products used in explorer 7 and explorer 8 are concizumab 40 mg/ml and concizumab 100 mg/ml drug products.

When patients are randomised/allocated to concizumab prophylaxis, they receive a loading dose of 1.0 mg/kg concizumab, followed by an initial daily dose of 0.20 mg/kg concizumab from treatment day 2. A potential dose adjustment to 0.25 mg/kg or 0.15 mg/kg concizumab, per day, takes place after 4 weeks, based on the concizumab exposure level. Patients who have concizumab exposure levels of greater than 4000 ng/ml will be dose adjusted to 0.15 mg/kg. Patients who have concizumab exposure levels of less than 200 ng/ml will be dose adjusted to 0.25 mg/kg. Patients who have concizumab exposure levels of 200-4000 ng/ml will continue to receive 0.20 mg/kg concizumab.

While certain features of the method disclosed herein have been illustrated and described herein, many modifications, substitutions, changes, and equivalents will now be evident to those of ordinary skill in the art. It is, therefore, to be understood that the appended claims are intended to cover all such modifications and changes as fall within the true spirit of the invention.

1. A method of preparing a pharmaceutical formulation of concizumab, comprising the following steps:
(a) obtaining or preparing a drug substance (DS) comprising concizumab as the active pharmaceutical ingredient and having a pH of 5.0-6.5;
(b) preparing an aqueous, surfactant-free excipient solution comprising one or two antimicrobial preservatives, and having a pH of 5.0-6.5;
(c) mixing the DS and the excipient solution;
(d) adding surfactant to the mixture of the DS and excipient solution; and
(e) optionally, adding water.

2. The method according to claim 1, wherein the one or two antimicrobial preservatives are selected from the group consisting of benzyl alcohol, chlorobutanol, m-cresol and phenol.

3. The method according to claim 1, wherein the surfactant is selected from the group consisting of polysorbate 20, polysorbate 80 and poloxamer 188, or a combination thereof.

4. The method according to claim 1, wherein:
the preservative is benzyl alcohol, in a concentration of less than 35 mg/ml in the DS-excipient mixture, and the surfactant subsequently added is Polysorbate 20 or Polysorbate 80; or
the preservative is benzyl alcohol, in a concentration of no more than 35 mg/ml in the DS-excipient mixture, and the surfactant subsequently added is Poloxamer 188; or
the preservative is chlorobutanol, in a concentration of no more than 7.5 mg/ml in the DS-excipient mixture, and the surfactant subsequently added is Polysorbate 20, Polysorbate 80 or Poloxamer 188; or
the preservative is m-cresol, in a concentration of less than 3 mg/ml in the DS-excipient mixture, and the surfactant subsequently added is Polysorbate 20; or
the preservative is m-cresol, in a concentration of less than 5 mg/ml, in the DS-excipient mixture, and the surfactant subsequently added is Polysorbate 80; or
the preservative is m-cresol, in a concentration of less than 3 mg/ml in the DS-excipient mixture, and the surfactant subsequently added is Polysorbate 80; or
the preservative is m-cresol, in a concentration of less than 10 mg/ml in the DS-excipient mixture, and the surfactant subsequently added is Poloxamer 188; or
the preservative is phenol, in a concentration of less than 6 mg/ml in the DS-excipient mixture, and the surfactant subsequently added is Polysorbate 20; or
the preservative is phenol, in a concentration of less than 10 mg/ml, in the DS-excipient mixture, and the surfactant subsequently added is Polysorbate 80; or
the preservative is phenol, in a concentration of less than 6 mg/ml in the DS-excipient mixture, and the surfactant subsequently added is Polysorbate 80; or
the preservative is phenol, in a concentration of less than 10 mg/ml in the DS-excipient mixture, and the surfactant subsequently added is Poloxamer 188.

5. The method according to claim 1, wherein: the excipient solution comprises phenol as a single preservative;
the concentration of phenol in the excipient solution is less than 55 mg/ml; and
the concentration of phenol in the DS-excipient mixture is less than 8 mg/ml; and
the surfactant is polysorbate 80, added to a final concentration of more than 0.1 mg/ml.

6. The method according to claim 1, wherein the drug substance and the aqueous, surfactant-free excipient solution comprise the same buffer(s), tonicity agent(s), viscosity lowering agent(s) and stabiliser(s).

7. The method according to claim 1, wherein the drug substance and the aqueous, surfactant-free excipient solution comprise the same buffer(s), tonicity agent(s) and salt(s).

8. The method according to claim 1, wherein the aqueous, surfactant-free excipient solution comprises histidine, sucrose, arginine hydrochloride and sodium chloride.

9. The pharmaceutical formulation obtained by the method according to claim 1.

10. The pharmaceutical formulation according to claim 9, comprising concizumab in a concentration of 5-110 mg/ml.

11. The pharmaceutical formulation according to claim 10, wherein the pharmaceutical formulation comprises 3-6 mg/ml phenol.

12. The pharmaceutical formulation according to claim 11, comprising:
0.1-2.0 mg/ml, polysorbate 20,
0.1-2.0 mg/ml, polysorbate 80 or
0.1-10 mg/ml, poloxamer 188.

13. The pharmaceutical formulation according to claim 9, comprising:
Concizumab: 5-110 mg/ml,
Phenol: about 0.35% (3.5 mg/ml),
L-Histidine: about 33 mM (5.12 mg/ml),
Sodium chloride: about 25 mM (1.46 mg/ml),
L-Arginine HCl: about 25 mM (5.27 mg/ml),
Sucrose: about 150 mM (51.3 mg/ml),
Polysorbate 80: about 0.25 mg/mL,
Water, and the formulation has a pH of about 6.0.

14. (canceled)

15. (canceled)

16. The method according to claim 2, wherein the surfactant is selected from the group consisting of polysorbate 20, polysorbate 80 and poloxamer 188, or a combination thereof.

17. The method according to claim 1, wherein:
the excipient solution comprises phenol as a single preservative;
the concentration of phenol in the excipient solution is less than 45 mg/ml; and
the concentration of phenol in the DS-excipient mixture is about 6 mg/ml; and
the surfactant is polysorbate 80, added to a final concentration of 0.1-2.0 mg/ml.

18. The pharmaceutical formulation obtained by the method according to claim 16.

19. The pharmaceutical formulation according to claim 9, comprising concizumab in a concentration of about 100 mg/ml.

20. The pharmaceutical formulation according to claim 10, wherein the pharmaceutical formulation comprises about 3.5 mg/ml phenol.

21. The pharmaceutical formulation according to claim 20, comprising:
0.1-0.3 mg/ml polysorbate 20,
About 0.25 mg/ml polysorbate 80 or
about 1.0 mg/ml poloxamer 188.

22. The pharmaceutical formulation according to claim 13, comprising about 100 mg/ml Concizumab.

23. A method of treating a coagulopathy, comprising administering to a subject in need thereof a therapeutically effective amount of the pharmaceutical formulation according to claim 22.

24. A method of treatment for the prophylaxis of a coagulopathy, comprising administering to a subject in need thereof a prophylactically effective amount of the pharmaceutical formulation according to claim 22.


SEQUENCE LISTING (2 sequences)

SEQ ID NO: 1 - protein, 219 aa, Artificial Sequence
  Represents the light chain of concizumab
DIVMTQTPLSLSVTPGQPASISCKSSQSLLESDGKTYLNWYLQKPGQSPQLLIYLVSILDSGVPDRFSGSGSGTDFTLKISRVEAEDVGVYYCLQATHFPQTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 448 aa, Artificial Sequence
  Represents the heavy chain of concizumab.
EVQLVESGGGLVKPGGSLRLSCAASGFTFSNYAMSWVRQTPEKRLEWVATISRSGSYSYFPDSVQGRFTISRDNAKNSLYLQMNSLRAEDTAVYYCARLGGYDEGDAMDSWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK